SEQUENCE LISTING (213 sequences)

SEQ ID NO: 1 - DNA, 17 bp, synthetic construct
  source 1..17: mol_type = other DNA
tgacctaagtgtaaagt

SEQ ID NO: 2 - DNA, 18 bp, synthetic construct
  source 1..18: mol_type = other DNA
tgagatttccttcaggtt

SEQ ID NO: 3 - DNA, 31 bp, synthetic construct
  source 1..31: mol_type = other DNA
tatatagttctgtatgagaccactctttccc

SEQ ID NO: 4 - DNA, 17 bp, synthetic construct
  source 1..17: mol_type = other DNA
accataaacgtgaaatg

SEQ ID NO: 5 - DNA, 18 bp, synthetic construct
  source 1..18: mol_type = other DNA
tctttggatttgggaatc

SEQ ID NO: 6 - DNA, 31 bp, synthetic construct
  source 1..31: mol_type = other DNA
ttataagttctgtatgagaccactctttccc

SEQ ID NO: 7 - DNA, 50 bp, synthetic construct
  source 1..50: mol_type = other DNA
vnanantvhanannttnnnatranattcngdgvgnaanatabtnctvgnd

SEQ ID NO: 8 - DNA, 50 bp, synthetic construct
  source 1..50: mol_type = other DNA
gcnggdggcgngttcbbctgtnvnagcntncgdngncgtttnncntcggt

SEQ ID NO: 9 - DNA, 10 bp, synthetic construct
  source 1..10: mol_type = other DNA
gaaatgcctt

SEQ ID NO: 10 - DNA, 10 bp, synthetic construct
  source 1..10: mol_type = other DNA
gtgggaacta

SEQ ID NO: 11 - DNA, 10 bp, synthetic construct
  source 1..10: mol_type = other DNA
gtgttgcttg

SEQ ID NO: 12 - DNA, 66 bp, synthetic construct
  source 1..66: mol_type = other DNA
tgacctaagtgtaaagttgagatttccttcaggtttatatagttctgtatgagaccactctttccc

SEQ ID NO: 13 - DNA, 66 bp, synthetic construct
  source 1..66: mol_type = other DNA
accataaacgtgaaatgtctttggatttgggaatcttataagttctgtatgagaccactctttccc

SEQ ID NO: 14 - DNA, 66 bp, synthetic construct
  source 1..66: mol_type = other DNA
tgacctaagtgtaaagtnnnnnnnnnnnnnnnnnnnnnnnnnnnnnnnnnnnnnnnnnnnnnnnnn

SEQ ID NO: 15 - DNA, 66 bp, synthetic construct
  source 1..66: mol_type = other DNA
nnnnnnnnnnnnnnnnntgagatttccttcaggttnnnnnnnnnnnnnnnnnnnnnnnnnnnnnnn

SEQ ID NO: 16 - DNA, 66 bp, synthetic construct
  source 1..66: mol_type = other DNA
nnnnnnnnnnnnnnnnnnnnnnnnnnnnnnnnnnntatatagttctgtatgagaccactctttccc

SEQ ID NO: 17 - DNA, 66 bp, synthetic construct
  source 1..66: mol_type = other DNA
tgacctaagtgtaaagttgagatttccttcaggttnnnnnnnnnnnnnnnnnnnnnnnnnnnnnnn

SEQ ID NO: 18 - DNA, 66 bp, synthetic construct
  source 1..66: mol_type = other DNA
nnnnnnnnnnnnnnnnntgagatttccttcaggtttatatagttctgtatgagaccactctttccc

SEQ ID NO: 19 - DNA, 66 bp, synthetic construct
  source 1..66: mol_type = other DNA
tgacctaagtgtaaagtnnnnnnnnnnnnnnnnnntatatagttctgtatgagaccactctttccc

SEQ ID NO: 20 - DNA, 66 bp, synthetic construct
  source 1..66: mol_type = other DNA
accataaacgtgaaatgnnnnnnnnnnnnnnnnnnnnnnnnnnnnnnnnnnnnnnnnnnnnnnnnn

SEQ ID NO: 21 - DNA, 66 bp, synthetic construct
  source 1..66: mol_type = other DNA
nnnnnnnnnnnnnnnnntctttggatttgggaatcnnnnnnnnnnnnnnnnnnnnnnnnnnnnnnn

SEQ ID NO: 22 - DNA, 66 bp, synthetic construct
  source 1..66: mol_type = other DNA
nnnnnnnnnnnnnnnnnnnnnnnnnnnnnnnnnnnttataagttctgtatgagaccactctttccc

SEQ ID NO: 23 - DNA, 66 bp, synthetic construct
  source 1..66: mol_type = other DNA
accataaacgtgaaatgtctttggatttgggaatcnnnnnnnnnnnnnnnnnnnnnnnnnnnnnnn

SEQ ID NO: 24 - DNA, 66 bp, synthetic construct
  source 1..66: mol_type = other DNA
nnnnnnnnnnnnnnnnntctttggatttgggaatcttataagttctgtatgagaccactctttccc

SEQ ID NO: 25 - DNA, 66 bp, synthetic construct
  source 1..66: mol_type = other DNA
accataaacgtgaaatgnnnnnnnnnnnnnnnnnnttataagttctgtatgagaccactctttccc

SEQ ID NO: 26 - DNA, 66 bp, synthetic construct
  source 1..66: mol_type = other DNA
tgacctaagtgtaaagttctttggatttgggaatcnnnnnnnnnnnnnnnnnnnnnnnnnnnnnnn

SEQ ID NO: 27 - DNA, 66 bp, synthetic construct
  source 1..66: mol_type = other DNA
tgacctaagtgtaaagtnnnnnnnnnnnnnnnnnnttataagttctgtatgagaccactctttccc

SEQ ID NO: 28 - DNA, 66 bp, synthetic construct
  source 1..66: mol_type = other DNA
nnnnnnnnnnnnnnnnntgagatttccttcaggttttataagttctgtatgagaccactctttccc

SEQ ID NO: 29 - DNA, 66 bp, synthetic construct
  source 1..66: mol_type = other DNA
accataaacgtgaaatgtgagatttccttcaggttnnnnnnnnnnnnnnnnnnnnnnnnnnnnnnn

SEQ ID NO: 30 - DNA, 66 bp, synthetic construct
  source 1..66: mol_type = other DNA
nnnnnnnnnnnnnnnnntctttggatttgggaatctatatagttctgtatgagaccactctttccc

SEQ ID NO: 31 - DNA, 66 bp, synthetic construct
  source 1..66: mol_type = other DNA
accataaacgtgaaatgnnnnnnnnnnnnnnnnnntatatagttctgtatgagaccactctttccc

SEQ ID NO: 32 - DNA, 66 bp, synthetic construct
  source 1..66: mol_type = other DNA
tgacctaagtgtaaagttctttggatttgggaatcttataagttctgtatgagaccactctttccc

SEQ ID NO: 33 - DNA, 66 bp, synthetic construct
  source 1..66: mol_type = other DNA
accataaacgtgaaatgtgagatttccttcaggttttataagttctgtatgagaccactctttccc

SEQ ID NO: 34 - DNA, 66 bp, synthetic construct
  source 1..66: mol_type = other DNA
accataaacgtgaaatgtctttggatttgggaatctatatagttctgtatgagaccactctttccc

SEQ ID NO: 35 - DNA, 66 bp, synthetic construct
  source 1..66: mol_type = other DNA
tgacctaagtgtaaagttgagatttccttcaggttttataagttctgtatgagaccactctttccc

SEQ ID NO: 36 - DNA, 66 bp, synthetic construct
  source 1..66: mol_type = other DNA
tgacctaagtgtaaagttctttggatttgggaatctatatagttctgtatgagaccactctttccc

SEQ ID NO: 37 - DNA, 66 bp, synthetic construct
  source 1..66: mol_type = other DNA
accataaacgtgaaatgtgagatttccttcaggtttatatagttctgtatgagaccactctttccc

SEQ ID NO: 38 - DNA, 10 bp, synthetic construct
  source 1..10: mol_type = other DNA
actctttccc

SEQ ID NO: 39 - DNA, 50 bp, synthetic construct
  source 1..50: mol_type = other DNA
ccatgatcccccactattaaggatatccggagaggatgctacctatcagg

SEQ ID NO: 40 - DNA, 50 bp, synthetic construct
  source 1..50: mol_type = other DNA
agaccagctgtatagcctcagaatgatccgtccggaaacccttactaggt

SEQ ID NO: 41 - DNA, 50 bp, synthetic construct
  source 1..50: mol_type = other DNA
gcatattgcagaacttgtgaagaggctttatgcgccacacactgcaagca

SEQ ID NO: 42 - DNA, 50 bp, synthetic construct
  source 1..50: mol_type = other DNA
gcaaaagcaaaagcattaagtagctttctggcataaacatattgcagctg

SEQ ID NO: 43 - DNA, 50 bp, synthetic construct
  source 1..50: mol_type = other DNA
ggttgcggtaagcattagagggctatcagcagcatcttatcgcagcggag

SEQ ID NO: 44 - DNA, 50 bp, synthetic construct
  source 1..50: mol_type = other DNA
ctgcagtataaccttgaagtaccatcacggagagaaacagtgccatctca

SEQ ID NO: 45 - DNA, 50 bp, synthetic construct
  source 1..50: mol_type = other DNA
aacgactacgtagtgttctataacaatcatgagaaattttagttctagat

SEQ ID NO: 46 - DNA, 50 bp, synthetic construct
  source 1..50: mol_type = other DNA
gctggtggcgagttccgctgtgccagcttccgttggcgtttgccatcggt

SEQ ID NO: 47 - DNA, 50 bp, synthetic construct
  source 1..50: mol_type = other DNA
atgcggccttgtgtggtggctcattgtggtgggccagaaaacgcgtgcaa

SEQ ID NO: 48 - DNA, 50 bp, synthetic construct
  source 1..50: mol_type = other DNA
gccagaggcgctggggccaggaggcgcaagccgggccctgaagccgcctc

SEQ ID NO: 49 - DNA, 50 bp, synthetic construct
  source 1..50: mol_type = other DNA
aaagaaattaggaaattcctgtggaagctggcgcgttgatgcacttcgtc

SEQ ID NO: 50 - DNA, 50 bp, synthetic construct
  source 1..50: mol_type = other DNA
caacaaacattttgcttttttaaaattgaaagaacaactgttttccgggt

SEQ ID NO: 51 - DNA, 67 bp, synthetic construct
  source 1..67: mol_type = other DNA
tgacctaagtgtaaagtvnanantvhanannttnnnatranattcngdgvgnaanatabtnctvgnd

SEQ ID NO: 52 - DNA, 67 bp, synthetic construct
  source 1..67: mol_type = other DNA
tgacctaagtgtaaagtgcnggdggcgngttcbbctgtnvnagcntncgdngncgtttnncntcggt

SEQ ID NO: 53 - DNA, 68 bp, synthetic construct
  source 1..68: mol_type = other DNA
tgagatttccttcaggttvnanantvhanannttnnnatranattcngdgvgnaanatabtnctvgnd

SEQ ID NO: 54 - DNA, 68 bp, synthetic construct
  source 1..68: mol_type = other DNA
tgagatttccttcaggttgcnggdggcgngttcbbctgtnvnagcntncgdngncgtttnncntcggt

SEQ ID NO: 55 - DNA, 81 bp, synthetic construct
  source 1..81: mol_type = other DNA
tatatagttctgtatgagaccactctttcccvnanantvhanannttnnnatranattcngdgvgnaanatabtnctvgnd

SEQ ID NO: 56 - DNA, 81 bp, synthetic construct
  source 1..81: mol_type = other DNA
tatatagttctgtatgagaccactctttcccgcnggdggcgngttcbbctgtnvnagcntncgdngncgtttnncntcggt

SEQ ID NO: 57 - DNA, 67 bp, synthetic construct
  source 1..67: mol_type = other DNA
accataaacgtgaaatgvnanantvhanannttnnnatranattcngdgvgnaanatabtnctvgnd

SEQ ID NO: 58 - DNA, 67 bp, synthetic construct
  source 1..67: mol_type = other DNA
accataaacgtgaaatggcnggdggcgngttcbbctgtnvnagcntncgdngncgtttnncntcggt

SEQ ID NO: 59 - DNA, 68 bp, synthetic construct
  source 1..68: mol_type = other DNA
tctttggatttgggaatcvnanantvhanannttnnnatranattcngdgvgnaanatabtnctvgnd

SEQ ID NO: 60 - DNA, 68 bp, synthetic construct
  source 1..68: mol_type = other DNA
tctttggatttgggaatcgcnggdggcgngttcbbctgtnvnagcntncgdngncgtttnncntcggt

SEQ ID NO: 61 - DNA, 81 bp, synthetic construct
  source 1..81: mol_type = other DNA
ttataagttctgtatgagaccactctttcccvnanantvhanannttnnnatranattcngdgvgnaanatabtnctvgnd

SEQ ID NO: 62 - DNA, 81 bp, synthetic construct
  source 1..81: mol_type = other DNA
ttataagttctgtatgagaccactctttcccgcnggdggcgngttcbbctgtnvnagcntncgdngncgtttnncntcggt

SEQ ID NO: 63 - DNA, 67 bp, synthetic construct
  source 1..67: mol_type = other DNA
tgacctaagtgtaaagtccatgatcccccactattaaggatatccggagaggatgctacctatcagg

SEQ ID NO: 64 - DNA, 67 bp, synthetic construct
  source 1..67: mol_type = other DNA
tgacctaagtgtaaagtagaccagctgtatagcctcagaatgatccgtccggaaacccttactaggt

SEQ ID NO: 65 - DNA, 67 bp, synthetic construct
  source 1..67: mol_type = other DNA
tgacctaagtgtaaagtgcatattgcagaacttgtgaagaggctttatgcgccacacactgcaagca

SEQ ID NO: 66 - DNA, 67 bp, synthetic construct
  source 1..67: mol_type = other DNA
tgacctaagtgtaaagtgcaaaagcaaaagcattaagtagctttctggcataaacatattgcagctg

SEQ ID NO: 67 - DNA, 67 bp, synthetic construct
  source 1..67: mol_type = other DNA
tgacctaagtgtaaagtggttgcggtaagcattagagggctatcagcagcatcttatcgcagcggag

SEQ ID NO: 68 - DNA, 67 bp, synthetic construct
  source 1..67: mol_type = other DNA
tgacctaagtgtaaagtctgcagtataaccttgaagtaccatcacggagagaaacagtgccatctca

SEQ ID NO: 69 - DNA, 67 bp, synthetic construct
  source 1..67: mol_type = other DNA
tgacctaagtgtaaagtaacgactacgtagtgttctataacaatcatgagaaattttagttctagat

SEQ ID NO: 70 - DNA, 67 bp, synthetic construct
  source 1..67: mol_type = other DNA
tgacctaagtgtaaagtgctggtggcgagttccgctgtgccagcttccgttggcgtttgccatcggt

SEQ ID NO: 71 - DNA, 67 bp, synthetic construct
  source 1..67: mol_type = other DNA
tgacctaagtgtaaagtatgcggccttgtgtggtggctcattgtggtgggccagaaaacgcgtgcaa

SEQ ID NO: 72 - DNA, 67 bp, synthetic construct
  source 1..67: mol_type = other DNA
tgacctaagtgtaaagtgccagaggcgctggggccaggaggcgcaagccgggccctgaagccgcctc

SEQ ID NO: 73 - DNA, 67 bp, synthetic construct
  source 1..67: mol_type = other DNA
tgacctaagtgtaaagtaaagaaattaggaaattcctgtggaagctggcgcgttgatgcacttcgtc

SEQ ID NO: 74 - DNA, 67 bp, synthetic construct
  source 1..67: mol_type = other DNA
tgacctaagtgtaaagtcaacaaacattttgcttttttaaaattgaaagaacaactgttttccgggt

SEQ ID NO: 75 - DNA, 68 bp, synthetic construct
  source 1..68: mol_type = other DNA
tgagatttccttcaggttccatgatcccccactattaaggatatccggagaggatgctacctatcagg

SEQ ID NO: 76 - DNA, 68 bp, synthetic construct
  source 1..68: mol_type = other DNA
tgagatttccttcaggttagaccagctgtatagcctcagaatgatccgtccggaaacccttactaggt

SEQ ID NO: 77 - DNA, 68 bp, synthetic construct
  source 1..68: mol_type = other DNA
tgagatttccttcaggttgcatattgcagaacttgtgaagaggctttatgcgccacacactgcaagca

SEQ ID NO: 78 - DNA, 68 bp, synthetic construct
  source 1..68: mol_type = other DNA
tgagatttccttcaggttgcaaaagcaaaagcattaagtagctttctggcataaacatattgcagctg

SEQ ID NO: 79 - DNA, 68 bp, synthetic construct
  source 1..68: mol_type = other DNA
tgagatttccttcaggttggttgcggtaagcattagagggctatcagcagcatcttatcgcagcggag

SEQ ID NO: 80 - DNA, 68 bp, synthetic construct
  source 1..68: mol_type = other DNA
tgagatttccttcaggttctgcagtataaccttgaagtaccatcacggagagaaacagtgccatctca

SEQ ID NO: 81 - DNA, 68 bp, synthetic construct
  source 1..68: mol_type = other DNA
tgagatttccttcaggttaacgactacgtagtgttctataacaatcatgagaaattttagttctagat

SEQ ID NO: 82 - DNA, 68 bp, synthetic construct
  source 1..68: mol_type = other DNA
tgagatttccttcaggttgctggtggcgagttccgctgtgccagcttccgttggcgtttgccatcggt

SEQ ID NO: 83 - DNA, 68 bp, synthetic construct
  source 1..68: mol_type = other DNA
tgagatttccttcaggttatgcggccttgtgtggtggctcattgtggtgggccagaaaacgcgtgcaa

SEQ ID NO: 84 - DNA, 68 bp, synthetic construct
  source 1..68: mol_type = other DNA
tgagatttccttcaggttgccagaggcgctggggccaggaggcgcaagccgggccctgaagccgcctc

SEQ ID NO: 85 - DNA, 68 bp, synthetic construct
  source 1..68: mol_type = other DNA
tgagatttccttcaggttaaagaaattaggaaattcctgtggaagctggcgcgttgatgcacttcgtc

SEQ ID NO: 86 - DNA, 68 bp, synthetic construct
  source 1..68: mol_type = other DNA
tgagatttccttcaggttcaacaaacattttgcttttttaaaattgaaagaacaactgttttccgggt

SEQ ID NO: 87 - DNA, 81 bp, synthetic construct
  source 1..81: mol_type = other DNA
tatatagttctgtatgagaccactctttcccccatgatcccccactattaaggatatccggagaggatgctacctatcagg

SEQ ID NO: 88 - DNA, 81 bp, synthetic construct
  source 1..81: mol_type = other DNA
tatatagttctgtatgagaccactctttcccagaccagctgtatagcctcagaatgatccgtccggaaacccttactaggt

SEQ ID NO: 89 - DNA, 81 bp, synthetic construct
  source 1..81: mol_type = other DNA
tatatagttctgtatgagaccactctttcccgcatattgcagaacttgtgaagaggctttatgcgccacacactgcaagca

SEQ ID NO: 90 - DNA, 81 bp, synthetic construct
  source 1..81: mol_type = other DNA
tatatagttctgtatgagaccactctttcccgcaaaagcaaaagcattaagtagctttctggcataaacatattgcagctg

SEQ ID NO: 91 - DNA, 81 bp, synthetic construct
  source 1..81: mol_type = other DNA
tatatagttctgtatgagaccactctttcccggttgcggtaagcattagagggctatcagcagcatcttatcgcagcggag

SEQ ID NO: 92 - DNA, 81 bp, synthetic construct
  source 1..81: mol_type = other DNA
tatatagttctgtatgagaccactctttcccctgcagtataaccttgaagtaccatcacggagagaaacagtgccatctca

SEQ ID NO: 93 - DNA, 81 bp, synthetic construct
  source 1..81: mol_type = other DNA
tatatagttctgtatgagaccactctttcccaacgactacgtagtgttctataacaatcatgagaaattttagttctagat

SEQ ID NO: 94 - DNA, 81 bp, synthetic construct
  source 1..81: mol_type = other DNA
tatatagttctgtatgagaccactctttcccgctggtggcgagttccgctgtgccagcttccgttggcgtttgccatcggt

SEQ ID NO: 95 - DNA, 81 bp, synthetic construct
  source 1..81: mol_type = other DNA
tatatagttctgtatgagaccactctttcccatgcggccttgtgtggtggctcattgtggtgggccagaaaacgcgtgcaa

SEQ ID NO: 96 - DNA, 81 bp, synthetic construct
  source 1..81: mol_type = other DNA
tatatagttctgtatgagaccactctttcccgccagaggcgctggggccaggaggcgcaagccgggccctgaagccgcctc

SEQ ID NO: 97 - DNA, 81 bp, synthetic construct
  source 1..81: mol_type = other DNA
tatatagttctgtatgagaccactctttcccaaagaaattaggaaattcctgtggaagctggcgcgttgatgcacttcgtc

SEQ ID NO: 98 - DNA, 81 bp, synthetic construct
  source 1..81: mol_type = other DNA
tatatagttctgtatgagaccactctttccccaacaaacattttgcttttttaaaattgaaagaacaactgttttccgggt

SEQ ID NO: 99 - DNA, 67 bp, synthetic construct
  source 1..67: mol_type = other DNA
accataaacgtgaaatgccatgatcccccactattaaggatatccggagaggatgctacctatcagg

SEQ ID NO: 100 - DNA, 67 bp, synthetic construct
  source 1..67: mol_type = other DNA
accataaacgtgaaatgagaccagctgtatagcctcagaatgatccgtccggaaacccttactaggt

SEQ ID NO: 101 - DNA, 67 bp, synthetic construct
  source 1..67: mol_type = other DNA
accataaacgtgaaatggcatattgcagaacttgtgaagaggctttatgcgccacacactgcaagca

SEQ ID NO: 102 - DNA, 67 bp, synthetic construct
  source 1..67: mol_type = other DNA
accataaacgtgaaatggcaaaagcaaaagcattaagtagctttctggcataaacatattgcagctg

SEQ ID NO: 103 - DNA, 67 bp, synthetic construct
  source 1..67: mol_type = other DNA
accataaacgtgaaatgggttgcggtaagcattagagggctatcagcagcatcttatcgcagcggag

SEQ ID NO: 104 - DNA, 67 bp, synthetic construct
  source 1..67: mol_type = other DNA
accataaacgtgaaatgctgcagtataaccttgaagtaccatcacggagagaaacagtgccatctca

SEQ ID NO: 105 - DNA, 67 bp, synthetic construct
  source 1..67: mol_type = other DNA
accataaacgtgaaatgaacgactacgtagtgttctataacaatcatgagaaattttagttctagat

SEQ ID NO: 106 - DNA, 67 bp, synthetic construct
  source 1..67: mol_type = other DNA
accataaacgtgaaatggctggtggcgagttccgctgtgccagcttccgttggcgtttgccatcggt

SEQ ID NO: 107 - DNA, 67 bp, synthetic construct
  source 1..67: mol_type = other DNA
accataaacgtgaaatgatgcggccttgtgtggtggctcattgtggtgggccagaaaacgcgtgcaa

SEQ ID NO: 108 - DNA, 67 bp, synthetic construct
  source 1..67: mol_type = other DNA
accataaacgtgaaatggccagaggcgctggggccaggaggcgcaagccgggccctgaagccgcctc

SEQ ID NO: 109 - DNA, 67 bp, synthetic construct
  source 1..67: mol_type = other DNA
accataaacgtgaaatgaaagaaattaggaaattcctgtggaagctggcgcgttgatgcacttcgtc

SEQ ID NO: 110 - DNA, 67 bp, synthetic construct
  source 1..67: mol_type = other DNA
accataaacgtgaaatgcaacaaacattttgcttttttaaaattgaaagaacaactgttttccgggt

SEQ ID NO: 111 - DNA, 68 bp, synthetic construct
  source 1..68: mol_type = other DNA
tctttggatttgggaatcccatgatcccccactattaaggatatccggagaggatgctacctatcagg

SEQ ID NO: 112 - DNA, 68 bp, synthetic construct
  source 1..68: mol_type = other DNA
tctttggatttgggaatcagaccagctgtatagcctcagaatgatccgtccggaaacccttactaggt

SEQ ID NO: 113 - DNA, 68 bp, synthetic construct
  source 1..68: mol_type = other DNA
tctttggatttgggaatcgcatattgcagaacttgtgaagaggctttatgcgccacacactgcaagca

SEQ ID NO: 114 - DNA, 68 bp, synthetic construct
  source 1..68: mol_type = other DNA
tctttggatttgggaatcgcaaaagcaaaagcattaagtagctttctggcataaacatattgcagctg

SEQ ID NO: 115 - DNA, 68 bp, synthetic construct
  source 1..68: mol_type = other DNA
tctttggatttgggaatcggttgcggtaagcattagagggctatcagcagcatcttatcgcagcggag

SEQ ID NO: 116 - DNA, 68 bp, synthetic construct
  source 1..68: mol_type = other DNA
tctttggatttgggaatcctgcagtataaccttgaagtaccatcacggagagaaacagtgccatctca

SEQ ID NO: 117 - DNA, 68 bp, synthetic construct
  source 1..68: mol_type = other DNA
tctttggatttgggaatcaacgactacgtagtgttctataacaatcatgagaaattttagttctagat

SEQ ID NO: 118 - DNA, 68 bp, synthetic construct
  source 1..68: mol_type = other DNA
tctttggatttgggaatcgctggtggcgagttccgctgtgccagcttccgttggcgtttgccatcggt

SEQ ID NO: 119 - DNA, 68 bp, synthetic construct
  source 1..68: mol_type = other DNA
tctttggatttgggaatcatgcggccttgtgtggtggctcattgtggtgggccagaaaacgcgtgcaa

SEQ ID NO: 120 - DNA, 68 bp, synthetic construct
  source 1..68: mol_type = other DNA
tctttggatttgggaatcgccagaggcgctggggccaggaggcgcaagccgggccctgaagccgcctc

SEQ ID NO: 121 - DNA, 68 bp, synthetic construct
  source 1..68: mol_type = other DNA
tctttggatttgggaatcaaagaaattaggaaattcctgtggaagctggcgcgttgatgcacttcgtc

SEQ ID NO: 122 - DNA, 68 bp, synthetic construct
  source 1..68: mol_type = other DNA
tctttggatttgggaatccaacaaacattttgcttttttaaaattgaaagaacaactgttttccgggt

SEQ ID NO: 123 - DNA, 81 bp, synthetic construct
  source 1..81: mol_type = other DNA
ttataagttctgtatgagaccactctttcccccatgatcccccactattaaggatatccggagaggatgctacctatcagg

SEQ ID NO: 124 - DNA, 81 bp, synthetic construct
  source 1..81: mol_type = other DNA
ttataagttctgtatgagaccactctttcccagaccagctgtatagcctcagaatgatccgtccggaaacccttactaggt

SEQ ID NO: 125 - DNA, 81 bp, synthetic construct
  source 1..81: mol_type = other DNA
ttataagttctgtatgagaccactctttcccgcatattgcagaacttgtgaagaggctttatgcgccacacactgcaagca

SEQ ID NO: 126 - DNA, 81 bp, synthetic construct
  source 1..81: mol_type = other DNA
ttataagttctgtatgagaccactctttcccgcaaaagcaaaagcattaagtagctttctggcataaacatattgcagctg

SEQ ID NO: 127 - DNA, 81 bp, synthetic construct
  source 1..81: mol_type = other DNA
ttataagttctgtatgagaccactctttcccggttgcggtaagcattagagggctatcagcagcatcttatcgcagcggag

SEQ ID NO: 128 - DNA, 81 bp, synthetic construct
  source 1..81: mol_type = other DNA
ttataagttctgtatgagaccactctttcccctgcagtataaccttgaagtaccatcacggagagaaacagtgccatctca

SEQ ID NO: 129 - DNA, 81 bp, synthetic construct
  source 1..81: mol_type = other DNA
ttataagttctgtatgagaccactctttcccaacgactacgtagtgttctataacaatcatgagaaattttagttctagat

SEQ ID NO: 130 - DNA, 81 bp, synthetic construct
  source 1..81: mol_type = other DNA
ttataagttctgtatgagaccactctttcccgctggtggcgagttccgctgtgccagcttccgttggcgtttgccatcggt

SEQ ID NO: 131 - DNA, 81 bp, synthetic construct
  source 1..81: mol_type = other DNA
ttataagttctgtatgagaccactctttcccatgcggccttgtgtggtggctcattgtggtgggccagaaaacgcgtgcaa

SEQ ID NO: 132 - DNA, 81 bp, synthetic construct
  source 1..81: mol_type = other DNA
ttataagttctgtatgagaccactctttcccgccagaggcgctggggccaggaggcgcaagccgggccctgaagccgcctc

SEQ ID NO: 133 - DNA, 81 bp, synthetic construct
  source 1..81: mol_type = other DNA
ttataagttctgtatgagaccactctttcccaaagaaattaggaaattcctgtggaagctggcgcgttgatgcacttcgtc

SEQ ID NO: 134 - DNA, 81 bp, synthetic construct
  source 1..81: mol_type = other DNA
ttataagttctgtatgagaccactctttccccaacaaacattttgcttttttaaaattgaaagaacaactgttttccgggt

SEQ ID NO: 135 - DNA, 17 bp, synthetic construct
  source 1..17: mol_type = other DNA
  misc_feature 8..10: note = anticodon
gcaggnnnnnnncctgt

SEQ ID NO: 136 - DNA, 18 bp, synthetic construct
  source 1..18: mol_type = other DNA
  misc_feature 8..11: note = 4nt anticodon
gcaggnnnnnnnncctgt

SEQ ID NO: 137 - DNA, 19 bp, synthetic construct
  source 1..19: mol_type = other DNA
  misc_feature 8..12: note = 5nt anticodon
gcaggnnnnnnnnncctgt

SEQ ID NO: 138 - DNA, 20 bp, synthetic construct
  source 1..20: mol_type = other DNA
  misc_feature 8..13: note = 6nt anticodon
gcaggnnnnnnnnnncctgt

SEQ ID NO: 139 - DNA, 17 bp, synthetic construct
  source 1..17: mol_type = other DNA
  misc_feature 8..10: note = anticodon
ttgggnnnnnnnctcaa

SEQ ID NO: 140 - DNA, 18 bp, synthetic construct
  source 1..18: mol_type = other DNA
  misc_feature 8..11: note = 4nt anticodon
ttgggnnnnnnnnctcaa

SEQ ID NO: 141 - DNA, 19 bp, synthetic construct
  source 1..19: mol_type = other DNA
  misc_feature 8..12: note = 5nt anticodon
ttgggnnnnnnnnnctcaa

SEQ ID NO: 142 - DNA, 20 bp, synthetic construct
  source 1..20: mol_type = other DNA
  misc_feature 8..13: note = 6nt anticodon
ttgggnnnnnnnnnnctcaa

SEQ ID NO: 143 - DNA, 17 bp, synthetic construct
  source 1..17: mol_type = other DNA
  misc_feature 8..10: note = anticodon
ttggannnnnnnttcaa

SEQ ID NO: 144 - DNA, 18 bp, synthetic construct
  source 1..18: mol_type = other DNA
  misc_feature 8..11: note = 4nt anticodon
ttggannnnnnnnttcaa

SEQ ID NO: 145 - DNA, 19 bp, synthetic construct
  source 1..19: mol_type = other DNA
  misc_feature 8..12: note = 5nt anticodon
ttggannnnnnnnnttcaa

SEQ ID NO: 146 - DNA, 20 bp, synthetic construct
  source 1..20: mol_type = other DNA
  misc_feature 8..13: note = 6nt anticodon
ttggannnnnnnnnnttcaa

SEQ ID NO: 147 - DNA, 17 bp, synthetic construct
  source 1..17: mol_type = other DNA
  misc_feature 8..10: note = anticodon
atggtnnnnnnnaccat

SEQ ID NO: 148 - DNA, 18 bp, synthetic construct
  source 1..18: mol_type = other DNA
  misc_feature 8..11: note = 4nt anticodon
atggtnnnnnnnnaccat

SEQ ID NO: 149 - DNA, 19 bp, synthetic construct
  source 1..19: mol_type = other DNA
  misc_feature 8..12: note = 5nt anticodon
atggtnnnnnnnnnaccat

SEQ ID NO: 150 - DNA, 20 bp, synthetic construct
  source 1..20: mol_type = other DNA
  misc_feature 8..13: note = 6nt anticodon
atggtnnnnnnnnnnaccat

SEQ ID NO: 151 - DNA, 17 bp, synthetic construct
  source 1..17: mol_type = other DNA
  misc_feature 8..10: note = anticodon
gcggannnnnnntccgc

SEQ ID NO: 152 - DNA, 18 bp, synthetic construct
  source 1..18: mol_type = other DNA
  misc_feature 8..11: note = 4nt anticodon
gcggannnnnnnntccgc

SEQ ID NO: 153 - DNA, 19 bp, synthetic construct
  source 1..19: mol_type = other DNA
  misc_feature 8..12: note = 5nt anticodon
gcggannnnnnnnntccgc

SEQ ID NO: 154 - DNA, 20 bp, synthetic construct
  source 1..20: mol_type = other DNA
  misc_feature 8..13: note = 6nt anticodon
gcggannnnnnnnnntccgc

SEQ ID NO: 155 - DNA, 17 bp, synthetic construct
  source 1..17: mol_type = other DNA
  misc_feature 8..10: note = anticodon
gcggtnnnnnnnaccgc

SEQ ID NO: 156 - DNA, 18 bp, synthetic construct
  source 1..18: mol_type = other DNA
  misc_feature 8..11: note = 4nt anticodon
gcggtnnnnnnnnaccgc

SEQ ID NO: 157 - DNA, 19 bp, synthetic construct
  source 1..19: mol_type = other DNA
  misc_feature 8..12: note = 5nt anticodon
gcggtnnnnnnnnnaccgc

SEQ ID NO: 158 - DNA, 20 bp, synthetic construct
  source 1..20: mol_type = other DNA
  misc_feature 8..13: note = 6nt anticodon
gcggtnnnnnnnnnnaccgc

SEQ ID NO: 159 - DNA, 17 bp, synthetic construct
  source 1..17: mol_type = other DNA
  misc_feature 8..10: note = anticodon
ggcggnnnnnnnccgcc

SEQ ID NO: 160 - DNA, 18 bp, synthetic construct
  source 1..18: mol_type = other DNA
  misc_feature 8..11: note = 4nt anticodon
ggcggnnnnnnnnccgcc

SEQ ID NO: 161 - DNA, 19 bp, synthetic construct
  source 1..19: mol_type = other DNA
  misc_feature 8..12: note = 5nt anticodon
ggcggnnnnnnnnnccgcc

SEQ ID NO: 162 - DNA, 20 bp, synthetic construct
  source 1..20: mol_type = other DNA
  misc_feature 8..13: note = 6nt anticodon
ggcggnnnnnnnnnnccgcc

SEQ ID NO: 163 - DNA, 17 bp, synthetic construct
  source 1..17: mol_type = other DNA
  misc_feature 8..10: note = anticodon
ggcgcnnnnnnngcgcc

SEQ ID NO: 164 - DNA, 18 bp, synthetic construct
  source 1..18: mol_type = other DNA
  misc_feature 8..11: note = 4nt anticodon
ggcgcnnnnnnnngcgcc

SEQ ID NO: 165 - DNA, 19 bp, synthetic construct
  source 1..19: mol_type = other DNA
  misc_feature 8..12: note = 5nt anticodon
ggcgcnnnnnnnnngcgcc

SEQ ID NO: 166 - DNA, 20 bp, synthetic construct
  source 1..20: mol_type = other DNA
  misc_feature 8..13: note = 6nt anticodon
ggcgcnnnnnnnnnngcgcc

SEQ ID NO: 167 - DNA, 17 bp, synthetic construct
  source 1..17: mol_type = other DNA
  misc_feature 8..10: note = anticodon
gcaggctnnnaacctgt

SEQ ID NO: 168 - DNA, 18 bp, synthetic construct
  source 1..18: mol_type = other DNA
  misc_feature 8..11: note = 4nt anticodon
gcaggctnnnnaacctgt

SEQ ID NO: 169 - DNA, 19 bp, synthetic construct
  source 1..19: mol_type = other DNA
  misc_feature 8..12: note = 5nt anticodon
gcaggctnnnnnaacctgt

SEQ ID NO: 170 - DNA, 20 bp, synthetic construct
  source 1..20: mol_type = other DNA
  misc_feature 8..13: note = 6nt anticodon
gcaggctnnnnnnaacctgt

SEQ ID NO: 171 - DNA, 17 bp, synthetic construct
  source 1..17: mol_type = other DNA
  misc_feature 8..10: note = anticodon
ttgggctnnnaactcaa

SEQ ID NO: 172 - DNA, 18 bp, synthetic construct
  source 1..18: mol_type = other DNA
  misc_feature 8..11: note = 4nt anticodon
ttgggctnnnnaactcaa

SEQ ID NO: 173 - DNA, 19 bp, synthetic construct
  source 1..19: mol_type = other DNA
  misc_feature 8..12: note = 5nt anticodon
ttgggctnnnnnaactcaa

SEQ ID NO: 174 - DNA, 20 bp, synthetic construct
  source 1..20: mol_type = other DNA
  misc_feature 8..13: note = 6nt anticodon
ttgggctnnnnnnaactcaa

SEQ ID NO: 175 - DNA, 17 bp, synthetic construct
  source 1..17: mol_type = other DNA
  misc_feature 8..10: note = anticodon
ttggactnnnaattcaa

SEQ ID NO: 176 - DNA, 18 bp, synthetic construct
  source 1..18: mol_type = other DNA
  misc_feature 8..11: note = 4nt anticodon
ttggactnnnnaattcaa

SEQ ID NO: 177 - DNA, 19 bp, synthetic construct
  source 1..19: mol_type = other DNA
  misc_feature 8..12: note = 5nt anticodon
ttggactnnnnnaattcaa

SEQ ID NO: 178 - DNA, 20 bp, synthetic construct
  source 1..20: mol_type = other DNA
  misc_feature 8..13: note = 6nt anticodon
ttggactnnnnnnaattcaa

SEQ ID NO: 179 - DNA, 17 bp, synthetic construct
  source 1..17: mol_type = other DNA
  misc_feature 8..10: note = anticodon
atggtctnnnaaaccat

SEQ ID NO: 180 - DNA, 18 bp, synthetic construct
  source 1..18: mol_type = other DNA
  misc_feature 8..11: note = 4nt anticodon
atggtctnnnnaaaccat

SEQ ID NO: 181 - DNA, 19 bp, synthetic construct
  source 1..19: mol_type = other DNA
  misc_feature 8..12: note = 5nt anticodon
atggtctnnnnnaaaccat

SEQ ID NO: 182 - DNA, 20 bp, synthetic construct
  source 1..20: mol_type = other DNA
  misc_feature 8..13: note = 6nt anticodon
atggtctnnnnnnaaaccat

SEQ ID NO: 183 - DNA, 17 bp, synthetic construct
  source 1..17: mol_type = other DNA
  misc_feature 8..10: note = anticodon
gcggactnnnaatccgc

SEQ ID NO: 184 - DNA, 18 bp, synthetic construct
  source 1..18: mol_type = other DNA
  misc_feature 8..11: note = 4nt anticodon
gcggactnnnnaatccgc

SEQ ID NO: 185 - DNA, 19 bp, synthetic construct
  source 1..19: mol_type = other DNA
  misc_feature 8..12: note = 5nt anticodon
gcggactnnnnnaatccgc

SEQ ID NO: 186 - DNA, 20 bp, synthetic construct
  source 1..20: mol_type = other DNA
  misc_feature 8..13: note = 6nt anticodon
gcggactnnnnnnaatccgc

SEQ ID NO: 187 - DNA, 17 bp, synthetic construct
  source 1..17: mol_type = other DNA
  misc_feature 8..10: note = anticodon
gcggtctnnnaaaccgc

SEQ ID NO: 188 - DNA, 18 bp, synthetic construct
  source 1..18: mol_type = other DNA
  misc_feature 8..11: note = 4nt anticodon
gcggtctnnnnaaaccgc

SEQ ID NO: 189 - DNA, 19 bp, synthetic construct
  source 1..19: mol_type = other DNA
  misc_feature 8..12: note = 5nt anticodon
gcggtctnnnnnaaaccgc

SEQ ID NO: 190 - DNA, 20 bp, synthetic construct
  source 1..20: mol_type = other DNA
  misc_feature 8..13: note = 6nt anticodon
gcggtctnnnnnnaaaccgc

SEQ ID NO: 191 - DNA, 17 bp, synthetic construct
  source 1..17: mol_type = other DNA
  misc_feature 8..10
ggcggctnnnaaccgcc

SEQ ID NO: 192 - DNA, 18 bp, synthetic construct
  source 1..18: mol_type = other DNA
  misc_feature 8..11: note = 4nt anticodon
ggcggctnnnnaaccgcc

SEQ ID NO: 193 - DNA, 19 bp, synthetic construct
  source 1..19: mol_type = other DNA
  misc_feature 8..12: note = 5nt anticodon
ggcggctnnnnnaaccgcc

SEQ ID NO: 194 - DNA, 20 bp, synthetic construct
  source 1..20: mol_type = other DNA
  misc_feature 8..13: note = 6nt anticodon
ggcggctnnnnnnaaccgcc

SEQ ID NO: 195 - DNA, 17 bp, synthetic construct
  source 1..17: mol_type = other DNA
  misc_feature 8..10: note = anticodon
ggcgcctnnnaagcgcc

SEQ ID NO: 196 - DNA, 18 bp, synthetic construct
  source 1..18: mol_type = other DNA
  misc_feature 8..11: note = 4nt anticodon
ggcgcctnnnnaagcgcc

SEQ ID NO: 197 - DNA, 19 bp, synthetic construct
  source 1..19: mol_type = other DNA
  misc_feature 8..12: note = 5nt anticodon
ggcgcctnnnnnaagcgcc

SEQ ID NO: 198 - DNA, 20 bp, synthetic construct
  source 1..20: mol_type = other DNA
  misc_feature 8..13: note = 6nt anticodon
ggcgcctnnnnnnaagcgcc

SEQ ID NO: 199 - DNA, 12 bp, synthetic construct
  source 1..12: mol_type = other DNA
tggggtttcccc

SEQ ID NO: 200 - DNA, 12 bp, synthetic construct
  source 1..12: mol_type = other DNA
aggggaaacccc

SEQ ID NO: 201 - DNA, 17 bp, synthetic construct
  source 1..17: mol_type = other DNA
  misc_feature 6..12: note = T loop
gcgggttcgaatcccgt

SEQ ID NO: 202 - DNA, 17 bp, synthetic construct
  source 1..17: mol_type = other DNA
  misc_feature 6..12: note = T loop
acgggttcgaatcccgt

SEQ ID NO: 203 - DNA, 17 bp, synthetic construct
  source 1..17: mol_type = other DNA
  misc_feature 6..12: note = T loop
gcgggttcgagtcccgt

SEQ ID NO: 204 - DNA, 17 bp, synthetic construct
  source 1..17: mol_type = other DNA
  misc_feature 6..12: note = T loop
acgggttcgagtcccgt

SEQ ID NO: 205 - DNA, 17 bp, synthetic construct
  source 1..17: mol_type = other DNA
  misc_feature 6..12: note = T loop
gtaggttcgagtcccat

SEQ ID NO: 206 - DNA, 17 bp, synthetic construct
  source 1..17: mol_type = other DNA
  misc_feature 6..12: note = T loop
gtcggttcgagtccgat

SEQ ID NO: 207 - DNA, 17 bp, synthetic construct
  source 1..17: mol_type = other DNA
  misc_feature 6..12: note = T loop
gccggttcgagtccggt

SEQ ID NO: 208 - DNA, 17 bp, synthetic construct
  source 1..17: mol_type = other DNA
  misc_feature 6..12: note = T loop
gcaggttcgagtcccgt

SEQ ID NO: 209 - DNA, 17 bp, synthetic construct
  source 1..17: mol_type = other DNA
  misc_feature 6..12: note = T loop
gccggttcgagtccggt

SEQ ID NO: 210 - RNA, 73 nt, synthetic construct
  source 1..73: mol_type = other RNA
  misc_feature 1..7: note = 5-prime part of acceptor stem
  misc_feature 10..13: note = 5-prime part of D stem
  misc_feature 22..25: note = 3-prime part of D stem
  misc_feature 27..31: note = 5-prime part of anticodon stem
  misc_feature 34..36: note = anticodon
  misc_feature 39..43: note = 3-prime part of anticodon stem
  misc_feature 49..53: note = 5-prime part of T stem
  misc_feature 61..65: note = 3-prime part of T stem
  misc_feature 66..72: note = 3-prime part of acceptor stem
ggctctgtggcgcaatggatagcgcattggacttcaaattcaaaggttgtgggttcgagtcccaccagagtcg

SEQ ID NO: 211 - RNA, 73 nt, synthetic construct
  source 1..73: mol_type = other RNA
  misc_feature 1..7: note = 5-prime part of acceptor stem
  misc_feature 10..13: note = 5-prime part of D stem
  misc_feature 27..31: note = 5-prime part of anticodon stem
  misc_feature 34..36: note = anticodon
  misc_feature 39..43: note = 3-prime part of anticodon stem
  misc_feature 49..53: note = 5-prime part of T stem
  misc_feature 61..65: note = 3-prime part of T stem
  misc_feature 66..72: note = 3-prime part of acceptor stem
  misc_feature 22..25: note = 3-prime part of D stem
ggctctgtggcgcaatggatagcgcattggacttcaaattcaaaggttgcgggttcgagtcccgtcagagtcg

SEQ ID NO: 212 - RNA, 91 nt, synthetic construct
  source 1..91: mol_type = other RNA
  misc_feature 1..7: note = 5-prime part of acceptor stem
  misc_feature 10..13: note = 5-prime part of D stem
  misc_feature 22..25: note = 3-prime part of D stem
  misc_feature 27..31: note = 5-prime part of anticodon stem
  misc_feature 34..36: note = anticodon
  intron 38..55
  misc_feature 57..61: note = 3-prime part of anticodon stem
  misc_feature 67..71: note = 5-prime part of T stem
  misc_feature 79..83: note = 3-prime part of T stem
  misc_feature 84..90: note = 3-prime part of acceptor stem
ggctctgtggcgcaatggatagcgcattggacttcaagctgagcctagtgtggtcattcaaaggttgcgggttcgagtcccgtcagagtcg

SEQ ID NO: 213 - RNA, 73 nt, synthetic construct
  source 1..73: mol_type = other RNA
  misc_feature 1..7: note = 5-prime part of acceptor stem
  misc_feature 10..13: note = 5-prime part of D stem
  misc_feature 22..25: note = 3-prime part of D stem
  misc_feature 34..36: note = anticodon
  misc_feature 27..31: note = 5-prime part of anticodon stem
  misc_feature 39..43: note = 3-prime part of anticodon stem
  misc_feature 49..53: note = 5-prime part of T stem
  misc_feature 61..65: note = 3-prime part of T stem
  misc_feature 66..72: note = 3-prime part of acceptor stem
ggcgctgtggcgcaatggatagcgcattggacttcaaattcaaaggttgcgggttcgagtcccgtcagcgtcg

(19) United States
(12) Patent Application Publication
(10) Pub. No.: US 2026/0275374 A1
(43) Pub. Date: Sep. 17, 2026

(54) SYNTHETIC DNA CONSTRUCT ENCODING TRANSFER RNA

(71) Applicant: Universitaet Hamburg, Hamburg (DE)

(72) Inventors: Zoya Ignatova, Hamburg (DE); Suki Albers-Fomenko, Hamburg (DE); Marcos Davyt, Hamburg (DE); Nikhil Bharti, Hamburg (DE)

(73) Assignee: Universitaet Hamburg, Hamburg (DE)

(21) Appl. No.: 19/472,680

(22) PCT Filed: Apr. 4, 2024
(86) PCT No.: PCT/EP2024/059191

§ 371 Date: Oct. 5, 2025

(30) Foreign Application Priority Data
Apr. 6, 2023 (EP) 23167017.5

(51) Int. Cl. C12N 15/85; A61K 31/711; A61K 48/00

(52) U.S. Cl. CPC C12N 15/85; A61K 31/711; A61K 48/005; C12N 2310/531

(57) ABSTRACT

A synthetic DNA construct comprising (A) a nucleic acid encoding a transfer RNA and (B) a 5′ leader sequence, the 5′ leader sequence comprising sequence motifs for controlling the expression level of the transfer RNA.

The invention relates to a synthetic DNA construct encoding tRNA, that can be used, for example, for the delivery of transfer RNA into cells, for example human cells.

Transfer ribonucleic acids (tRNAs) are an essential part of the protein synthesis machinery of living cells as necessary components for translating the nucleotide sequence of a messenger RNA (mRNA) into the amino acid sequence of a protein. Naturally occurring tRNAs comprise an amino acid binding stem being able to covalently bind an amino acid and an anticodon loop containing a base triplet called “anticodon”, which can bind non-covalently to a corresponding base triplet called “codon” on an mRNA. A protein is synthesized by assembling the amino acids carried by tRNAs using the codon sequence on the mRNA as a template with the aid of a multi component system comprising, inter alia, the ribosome and several auxiliary enzymes.

Transfer RNAs have recently seen increasing interest in their use as drugs for therapeutic purposes, e.g. as part of a gene therapy for treating conditions associated with nonsense mutations, i.e. mutations changing a sense codon encoding one of the twenty amino acids specified by the genetic code to a chain-terminating codon (“premature termination codon”, PTC) in a gene sequence (Ai-Ming Yu, Young Hee Choi and Mei-Juan Tu, RNA Drugs and RNA Targets for Small Molecules: Principles, Progress, and Challenges, Pharmacological Reviews, 2020, 72 (4) 862-898; DOI: 10.1124/pr.120.019554; Porter, JJ, Heil, CS, Lueck, JD, Therapeutic promise of engineered nonsense suppressor tRNAs, WIREs RNA. 2021; 12: e1641, DOI: 10.1002/wrna.1641). Lueck et al. 2016 (Lueck, J. D., Infield, DT, Mackey, A L, Pope, R M, McCray, PB, Ahern, CA. Engineered tRNA suppression of a CFTR nonsense mutation, bioRxiv 088690; doi: 10.1101/088690), for example, describe a codon-edited tRNA enabling the conversion of an in-frame stop codon in the CFTR gene to the naturally occurring amino acid in order to restore the full-length wild type protein.

Compared to mRNA, tRNA molecules offer significantly higher stability and are on average 10-fold shorter, alleviating the problem of introduction into the target tissue. This has led to attempts to use tRNA in gene therapy in order to prevent the formation of a truncated protein from an mRNA with a premature stop codon and to introduce the correct amino acid instead (see, e.g., Koukuntla, R., 2009, Suppressor tRNA mediated gene therapy, Graduate Theses and Dissertations, 10920, Iowa State University, http://lib.dr.iastate.edu/etd/10920; US 2003/0224479 A1; U.S. Pat. No. 6,964,859). WO 2017/121863 A1 and WO 2020/208169 A1 describe synthetic tRNA with an extended anticodon loop that can, for example, be used as a suppressor tRNA for genetic diseases associated with a frameshift mutation. WO 2021/113218 A1 discloses engineered tRNA molecules and vectors encoding engineered suppressor tRNA molecules that recognize and readthrough of disease-causing premature stop codons. The use of tRNA in the context of gene therapy, e.g., of diseases associated with the existence of premature termination codons (PTCs), for example, is also described, inter alia, in WO 2020/069194 A1, WO 2021/211762 A2, WO 2021/087401 A1, WO 2021/113218 A1, and US 2020/291401 A1.

The biogenesis of tRNA comprises several processes, including transcription, 5′ and 3′ ends processing, splicing, post-transcriptional nucleotide modification, CCA addition and aminoacylation. The transcription of tRNA involves binding of the transcription factor TFIIIC to intragenic sequence motifs (promoters), called “A box” and “B box”, coding for parts of the D- and T arm, respectively, and the recruitment of the transcription factor TFIIIB to the 5′-upstream region of the tRNA gene, which directs recruitment of RNA polymerase III (pol III) transcribing the tRNA gene (see, for example, Kirchner, S., Ignatova, Z. Emerging roles of tRNA in adaptive translation, signalling dynamics and disease, Nat Rev Genet 16, 98-112 (2015), doi: 10.1038/nrg3861; Schramm L, Hernandez N., Recruitment of RNA polymerase III to its target promoters, Genes Dev. 2002 Oct. 15; 16 (20): 2593-620, doi: 10.1101/gad.1018902; Mitra S., Das P., Samadder A., Das S., Betai R., Chakrabarti J., 2015, Eukaryotic tRNAs fingerprint invertebrates vis-à-vis vertebrates, Journal of Biomolecular Structure and Dynamics, doi: 10.1080/07391102.2014.990925; Maraia, R. J., Arimbasseri, A. G., It's Sno′ing on Pol III at nuclear pores. Genome Biol 14, 137, 2013, doi: 10.1186/gb4137; Canella D, Bernasconi D, Gilardi F, LeMartelot G, Migliavacca E, Praz V, Cousin P, Delorenzi M, Hernandez N; CycliX Consortium. A multiplicity of factors contributes to selective RNA polymerase III occupancy of a subset of RNA polymerase III genes in mouse liver. Genome Res. 2012 April; 22 (4): 666-80. doi: 10.1101/gr.130286.111; Enserink J M, Chymkowitch P. Cell Cycle-Dependent Transcription: The Cyclin Dependent Kinase Cdk1 Is a Direct Regulator of Basal Transcription Machineries. Int J Mol Sci. 2022 Jan. 24; 23 (3): 1293, doi: 10.3390/ijms23031293; Berg M D, Brandl C J., Transfer RNAs: diversity in form and function, RNA Biol. 2021 March; 18 (3): 316-339, doi: 10.1080/15476286.2020.1809197; Korde A., Extra-Transcriptional Effects of Chromatin Bound RNA Polymerase III Transcription Complexes, 2014. LSU Doctoral Dissertations. 274, https://digitalcommons.lsu.edu/gradschool_dissertations/274, doi: 10.31390/gradschool_dissertations.274; Young L S, Rivier D H, Sprague K U. Sequences far downstream from the classical tRNA promoter elements bind RNA polymerase III transcription factors, Mol Cell Biol. 1991 March; 11 (3): 1382-92, doi: 10.1128/mcb.11.3.1382-1392.1991;). Zhang et al. 2011 (Zhang G, Lukoszek R, Mueller-Roeber B, Ignatova Z. Different sequence signatures in the upstream regions of plant and animal tRNA genes shape distinct modes of regulation. Nucleic Acids Res. 2011 April; 39 (8): 3331-9. doi: 10.1093/nar/gkq1257) describe anticodon-dependent sequence motifs within a non-coding 5′-upstream region of tRNA genes that might be involved in the regulation of tRNA transcription.

It is an object of the invention to improve the means and possibilities for gene therapy of diseases using transfer RNA. In particular, it is an object of the invention, to provide improved means for the delivery of suppressor tRNA to living cells, for example human cells.

For solving the problem, the invention provides, in one aspect, a synthetic DNA construct comprising (A) a nucleic acid encoding a transfer RNA and (B) a 5′ leader sequence, the 5′ leader sequence comprising
a) a sequence motif selected from the group consisting of the sequence motifs TGACCTAAGTGTAAAGT, I_{H}, (SEQ ID NO: 1), TGAGATTTCCTTCAGGTT, II_{H}, (SEQ ID NO: 2), TATATAGTTCTGTATGAGACCACTCTTTCCC, III_{H}, (SEQ ID NO: 3), ACCATAAACGTGAAATG, I_{L}, (SEQ ID NO: 4), TCTTTGGATTTGGGAATC, II_{L}, (SEQ ID NO: 5, and TTATAAGTTCTGTATGAGACCACTCTTTCCC, III_{L}, (SEQ ID NO: 6); and/or
b) a sequence motif selected from the group consisting of the sequence motifs VNANANTVHANANNTTNNNATRANATTCNGDGVGNAANATABTNCTVGND, 50 nt_{H}, (SEQ ID NO:7) and GCNGGDGGCGNGTTCBBCTGTNVNAGCNTNCGDNGNCGTTTNNCNTCGGT, 50 nt_{L}, (SEQ ID NO: 8); and/or
c) a sequence motif selected from the group consisting of the sequence motifs GAAATGCCTT, 10 nt_{H1}, (SEQ ID NO: 9), GTGGGAACTA, 10 nt_{H2}, (SEQ ID NO: 10), and GTGTTGCTTG, 10 nt_{H3}, (SEQ ID NO: 11).

The invention provides a novel synthetic DNA construct comprising a nucleic acid encoding a transfer RNA. The DNA construct can, for example, be configured as a gene delivery vehicle (GDV) for the delivery of transfer RNA, for example suppressor tRNA, into cells, in particular mammalian cells, e.g., human cells. Besides a tRNA gene, the synthetic DNA construct of the invention comprises a 5′ leader sequence functionally linked to the nucleic acid encoding the tRNA (tRNA gene), wherein the 5′ leader sequence comprises sequence motifs for the binding of the transcription factor TFIIIB. The invention provides sequence motifs having promoter activity that can be included in a 5′ leader sequence of a tRNA encoded downstream of the 5′ leader sequence. In preferred embodiments, additional sequence motifs, namely A and/or B box sequence motifs within the transfer RNA encoded downstream the 5′ leader sequence can be included in order to further enhance or better control binding of transcription factor TFIIIC. Suitable selection and, as the case may be, combination of the sequence motifs enables control of the transcription of the downstream tRNA gene. The tRNA encoded by the nucleic acid in the construct may, for example, be an engineered tRNA, having, for example, a modified anticodon being able to base-pair with a stop codon on an mRNA, and/or a modified T arm comprising a B box sequence motif. The B box sequence may, for example, be a sequence enhancing the binding of the transcription factor TFIIIC to the tRNA gene.

The terms “DNA construct” or “synthetic DNA construct” relate to an artificially-designed segment of DNA that can, for example, be used to incorporate genetic material into a target tissue or cell. The terms “vector”, “synthetic vector” or “synthetic gene delivery vehicle (GDV)” refer to any means for delivering a nucleic acid, for example a coding nucleic acid together with regulatory elements like promoter sequences and termination signals, into a living cell. Many viral and non-viral vectors are known. Examples are plasmids, viruses, cationic liposomes or polymers etc.

For the term “nucleic acid encoding a transfer RNA” the terms “transfer RNA gene” or “tRNA gene” may also be used synonymously.

The term “5′ leader sequence” as used herein refers to a sequence of nucleotides located “upstream” of the 5′ end of a tRNA gene, for example approximately 100 nt upstream of the 5′ end of the coding sequence for the mature tRNA, comprising a nucleotide sequence functioning as a binding site for the transcription factor TFIIIB. The term “5′ leader sequence” refers in particular to the sequence of nucleotides comprising or consisting of positions −100 to −15′ from the transcription start of a tRNA gene. “Mature tRNA” refers to a fully processed and functional tRNA. The terms “extragenic leader sequence”, “5′ untranslated region (5′ UTR) or “extragenic pol III binding sequence” may also be used here.

The terms “A box” and “B box” refer to intragenic (gene internal) regions, i.e., sequence motifs of a tRNA gene comprising nucleotide sequences to which the transcription factor TFIIIC of the RNA polymerase III binds. The boxes may also be considered as part of a tRNA promoter (so-called type 2 promoter) or promoter elements. The nucleotide sequence of about 10-14 nucleotides in length (Mitra S., Das P., Samadder A., Das S., Betai R., Chakrabarti J., 2015, Eukaryotic tRNAs fingerprint invertebrates vis-à-vis vertebrates, Journal of Biomolecular Structure and Dynamics, doi: 10.1080/07391102.2014.990925) forming the A box are located in the region of the tRNA gene encoding part of the D arm, the nucleotide sequence of about 11 nucleotides in length (Mitra S., Das P., Samadder A., Das S., Betai R., Chakrabarti J., 2015, Eukaryotic tRNAs fingerprint invertebrates vis-à-vis vertebrates, Journal of Biomolecular Structure and Dynamics, doi: 10.1080/07391102.2014.990925) forming the B box are located in the region of the tRNA gene encoding part of the T loop. An 11 nt consensus sequence for the B-Box is given by Mitra et al. 2015 (see above) as being RGTTCRANNCY, covering the nucleotides N52-N62 of the mature tRNA. Any numbering in relation to a tRNA gene or part thereof, e.g., the A and B boxes, refers to the nucleotide numbering within the mature tRNA, following the tRNA numbering convention (see below).

The term “encoding” in relation to a nucleotide sequence of a tRNA gene means that the nucleotide sequence is transcribed into a tRNA or part of a tRNA. A direct reference to a tRNA encoded by the DNA construct of the invention, e.g., a reference to a structural part of the mature tRNA, for example, the D arm, anticodon arm, T arm or acceptor stem, is to be understood as referring to the regions of the tRNA gene in the nucleic acid encoding those structural parts, unless clearly stated otherwise or clearly recognizable from the context. A wording according to which an encoded tRNA comprises, for example, a T arm of a specific sequence, the sequence presented as a DNA (with the nucleotides A, C, G, and T), is thus to be understood as referring to the sequence within the tRNA gene coding for the corresponding structure, wherein the corresponding structure appears in the mature tRNA transcribed from the tRNA gene, however, with T replaced by U, and, as the case may be, with modified nucleotides.

The term “sequence motif” or “sequence signature” refers to a specific sequence or consensus sequence having a particular function, e.g., as a binding site for an RNA polymerase.

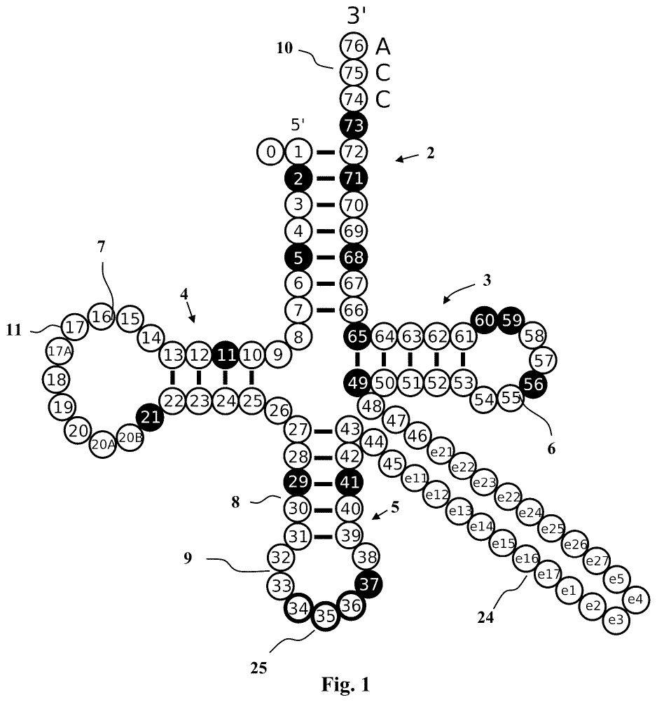

The terms “transfer ribonucleic acid” or “tRNA” refer to RNA molecules with a length of typically 73 to 90 nucleotides, which mediate the translation of a nucleotide sequence in a messenger RNA into the amino acid sequence of a protein. tRNAs are able to covalently bind a specific amino acid at their 3′ CCA tail at the end of the acceptor stem, and to base-pair via a usually three-nucleotide anticodon in the anticodon loop of the anticodon arm with a usually three-nucleotide sequence (codon) in the messenger RNA. Some anticodons can pair with more than one codon due to a phenomenon known as wobble base pairing. The secondary “cloverleaf” structure of tRNA comprises the acceptor stem binding the amino acid and three arms (“D arm”, “T arm” and “anticodon arm”) ending in loops (D loop, T loop (TψC loop), anticodon loop), i.e. sections with unpaired nucleotides. The terms “D stem”, “T stem” (or “TψC stem”) and “anticodon stem” (also “AC stem”) relate to portions of the D arm, T arm and anticodon arm, respectively, with paired nucleotides. Aminoacyl tRNA synthetases charge (aminoacylate) tRNAs with a specific amino acid. Each tRNA contains a distinct anticodon triplet sequence that can base-pair to one or more codons for an amino acid. By convention, the nucleotides of tRNAs are often numbered 1 to 76, starting from the 5′-phosphate terminus, based on a “consensus” tRNA molecule consisting of 76 nucleotides, and regardless of the actual number of nucleotides in the tRNA, which are not always of a length of 76 nt due to variable portions, such as the D loop or the variable loop in the tRNA (see FIG. 1). Following this convention, nucleotide positions 34-36 of naturally occurring tRNA refer to the three nucleotides of the anticodon, and positions 74-76 refer to the terminating CCA tail. Any “supernumerary” nucleotide can be numbered by adding alphabetic characters to the number of the previous nucleotide being part of the consensus tRNA and numbered according to the convention, for example 20a, 20b etc. for the D-arm, or by independently numbering the nucleotides and adding a leading letter, as in case of the variable loop such as e11, e12 etc. (see, for example, Sprinzl M, Horn C, Brown M, Ioudovitch A, Steinberg S. Compilation of tRNA sequences and sequences of tRNA genes. Nucleic Acids Res. 1998; 26 (1): 148-53). In the following, the tRNA-specific numbering will also be referred to as “tRNA numbering convention” or “transfer RNA numbering convention”.

The term “tRNA body” refers to the portion of the tRNA outside the anticodon loop.

The term “intron” relates to a polynucleotide sequence in a nucleic acid that is part of the nucleic acid within a genome and within a first nucleic acid product transcribed from the genomic nucleic acid, but is not contained in the final nucleic acid product. In case of a protein gene, for example, an intron is a non-coding polynucleotide sequence separating coding polynucleotide sequences (exons), which is excised from the pre-mRNA transcribed from the protein gene in a process called “splicing”. In case of a transfer RNA, for example, the term intron relates to a polynucleotide sequence that is excised (spliced) from a pre tRNA transcribed from the tRNA gene.

The term “intronic tRNA” relates to a tRNA, the precursor (pre-tRNA) of which contains an intron that is spliced from the pre-tRNA during processing of the pre-tRNA into the final (mature) tRNA. The term “non-intronic tRNA” relates to tRNA being generated from pre-tRNAs that do not contain an intron and do not undergo splicing. The terms “intronic tRNA” or “non-intronic tRNA” encompass the pre-tRNA and the mature tRNA.

The term “tDNA”, if used herein, relates to a sequence of DNA encoding a tRNA, in particular a DNA sequence having the sequence of a tRNA wherein the uracil nucleotides (U) of the tRNA are replaced with thymine nucleotides (T).

The terms “engineered transfer ribonucleic acid” or “synthetic tRNA” refer to a tRNA modified by chemical or molecular biological methods or a non-naturally occurring tRNA. The terms “engineered” and “synthetic” are used synonymously here. An example is a tRNA that will be aminoacylated with an amino acid under natural conditions, but has an anticodon pairing to a stop codon instead of the anticodon for the corresponding amino acid.

The term “codon” refers to a sequence of nucleotide triplets, i.e. three DNA or RNA nucleotides, corresponding to a specific amino acid or stop signal during protein synthesis. A list of codons (on mRNA level) and the encoded amino acids are given in the following:

| Amino acid | One Letter Code | Codons |
|---|---|---|
| Ala | A | GCU, GCC, GCA, GCG |
| Arg | R | CGU, CGC, CGA, CGG, AGA, AGG |
| Asn | N | AAU, AAC |
| Asp | D | GAU, GAC |
| Cys | C | UGU, UGC |
| Gln | Q | CAA, CAG |
| Glu | E | GAA, GAG |
| Gly | G | GGU, GGC, GGA, GGG |
| His | H | CAU, CAC |
| Ile | I | AUU, AUC, AUA |
| Leu | L | UUA, UUG, CUU, CUC, CUA, CUG |
| Lys | K | AAA, AAG |
| Met | M | AUG |
| Phe | F | UUU, UUC |
| Pro | P | CCU, CCC, CCA, CCG |
| Ser | S | UCU, UCC, UCA, UCG, AGU, AGC |
| Thr | T | ACU, ACC, ACA, ACG |
| Trp | W | UGG |
| Tyr | Y | UAU, UAC |
| Val | V | GUU, GUC, GUA, GUG |

START: AUG
STOP: UAA, UGA, UAG, abbreviated “X”

The term “sense codon” as used herein refers to a codon coding for an amino acid. The term “stop codon” or “nonsense codon” refers to a codon, i.e. a nucleotide triplet, of the genetic code not coding for one of the 20 amino acids normally found in proteins and signalling the termination of translation of a messenger RNA.

The term “frameshift mutation” refers to an out-of-frame insertion or deletion (collectively called “indels”) of nucleotides with a number not evenly divisible by three. This perturbs the nucleotide sequence decoding which proceeds in steps of three nucleotide bases. The term “−1 frameshift mutation” relates to the deletion of a single nucleotide causing a shift in the reading frame by one nucleotide leading to the first nucleotide of the following codon being read as part of the codon from which the nucleotide has been deleted. Deletion of one nucleotide from the upstream codon along with several triplets (i.e. deletion of 4, 7, 10 etc. nucleotides) is also considered as −1 frameshifting. The term “+1 frameshift mutation” relates to the insertion of a single nucleotide into a triplet, or the deletion of two nucleotides. The result of either event is to shift the reading frame by one nucleotide, such that a nucleotide of an upstream codon is being read as part of a downstream codon. Insertion of one nucleotide along with several triplets (3n+1 nucleotides, n being an integer, i.e. insertion of 4, 7, 10 etc. nucleotides), or deletion of two nucleotides from the upstream codon along with several triplets (i.e. deletion of 5, 8, 11 etc nucleotides) is also considered as +1 frameshifting.

The term “anticodon” refers to a sequence of usually three nucleotides within a tRNA that base-pair (non-covalently bind) to the three bases (nucleotides) of the codon on the mRNA. In a natural tRNA a standard three-nucleotide anticodon is usually represented by the nucleotides at positions 34, 35 and 36. An anticodon may also contain nucleotides with modified bases. The terms “four nucleotide anticodon” or “four base anticodon” relate to an anticodon having four consecutive nucleotides (bases) which pair with four consecutive bases on an mRNA. The terms “quadruplet nucleotide anticodon” or “quadruplet anticodon” may also be used to denote a “four nucleotide anticodon”. The terms “five nucleotide anticodon” or “five base anticodon” relate to an anticodon having five consecutive nucleotides (bases) which bind (base-pair) to five consecutive bases on an mRNA. The terms “quintuplet nucleotide anticodon” or “quintuplet anticodon” may also be used to denote a “five nucleotide anticodon”.

The term “anticodon arm” refers to a part of the tRNA comprising the anticodon. The anticodon arm is composed of a stem portion (“anticodon stem”), usually consisting of five base pairs (positions 27-31 and 39-43), and a loop portion (“anticodon loop”), i.e. a consecutive sequence of unpaired nucleotides bound to the anticodon stem. The anticodon arm takes the positions 27 to 43, the position numbering following transfer RNA numbering convention.

The term “anticodon loop” refers to the unpaired nucleotides of the anticodon arm containing the anticodon. Naturally occurring tRNAs usually have seven nucleotides in their anticodon loop, three of which pair to the codon in the mRNA.

The term “extended anticodon loop” refers to an anticodon loop with a higher number of nucleotides in the loop than in naturally occurring tRNAs. An extended anticodon loop may, for example, contain more than seven nucleotides, e.g. eight, nine, or ten nucleotides. In particular, the term relates to an anticodon loop comprising an anticodon composed of more than three consecutive nucleotides, e.g. four, five or six nucleotides, being able to base-pair with a corresponding number of consecutive nucleotides in an mRNA.

The term “anticodon stem” refers to the paired nucleotides of the anticodon arm that carry the anticodon loop.

The term “T arm” relates to a portion of a tRNA composed of a stem portion (“T stem”) and a loop portion (“T loop”) between the acceptor stem and the variable loop. The T arm takes the positions 49 to 65, the position numbering following transfer RNA numbering convention. The T stem is usually composed of five nucleotide pairs (taking positions 49-53 and 61-65).

The term “T stem” (or “TψC” stem) relates to the paired nucleotides of the T arm, which carry the T loop, i.e. the unpaired nucleotides of the T arm.

The term “D arm” relates to the tRNA portion composed of a stem (“D stem”), i.e. the paired nucleotides of the D arm, and a D loop, i.e. the unpaired nucleotides of the D arm, between the acceptor stem and the anticodon arm. The D arm takes the positions 10-25, the position numbering following transfer RNA numbering convention.

The term “variable loop” refers to a tRNA loop located between the anticodon arm and the T arm. The number of nucleotides composing the variable loop may largely vary from tRNA to tRNA. The variable loop can thus be rather short or even missing, or rather large, forming, for example, a helix. The term “variable arm” may synonymously be used for the term “variable loop”. Use of the term “variable loop” does not exclude the existence of a “stem” portion within the variable loop, i.e. a stretch of consecutive nucleotides of the variable loop forming base pairs with a complementary stretch of other consecutive nucleotides of the variable loop. The variable loop takes the positions 44 to 48, the position numbering following transfer RNA numbering convention. It should be noted that the variable loop may have a considerable number of additional nucleotides that are separately numbered (see FIG. 1).

The term “acceptor stem” relates to the site of attachment of amino acids to the tRNA. An acceptor stem is often formed by 7 base pairs.

The terms “codon base triplet” or “anticodon base triplet”, when used herein, refer to sequences of three consecutive nucleotides which form a codon or anticodon. Synonymously, the terms “three nucleotide codon” (also “three base codon”) or “three nucleotide anticodon” (also “three base anticodon”), or abbreviations thereof, e.g. “3 nt codon” or “3 nt anticodon”, may be used.

The term “base pair” refers to a pair of bases, or the formation of such a pair of bases, joined by hydrogen bonds. The term “Watson-Crick base pair” may also be used for such a pair of bases. One of the bases of the base pair is usually a purine, and the other base is usually a pyrimidine. In RNA, for example, the bases adenine and uracil can form a base pair and the bases guanine and cytosine can form a base pair. In DNA thymine usually forms a base pair with adenine instead of uracil. However, the formation of other base pairs (“wobble base pairs”) is also possible, e.g. base pairs of guanine-uracil (G-U), hypoxanthine-uracil (I-U), hypoxanthine-adenine (I-A), and hypoxanthine-cytosine (I-C). The term “being able to base-pair” refers to the ability of nucleotides or sequences of nucleotides to form hydrogen-bond-stabilized structures with a corresponding nucleotide or nucleotide sequence. Base pairing can occur intramolecular, e.g., within a single-stranded nucleic acid (e.g., a tRNA), or intermolecular, i.e., between different nucleic acid molecules.

The term “base”, as used herein, for example in terms like “the bases A, C, G or U” or “the bases A, C, G or T”, encompasses or is synonymously used to the term “nucleotide”, unless the context clearly indicates otherwise.

Abbreviations used her for bases or nucleotides are according to the IUPAC nucleotide code and standard ST.26 (version 1.5), as follows:

| IUPAC nucleotide code | Base |
|---|---|
| A | Adenine |
| C | Cytosine |
| G | Guanine |
| T (or U) | Thymine (or Uracil in RNA) |
| R | A or G |
| Y | C or T/U |
| S | G or C |
| W | A or T/U |
| K | G or T/U |
| M | A or C |
| B | C or G or T/U |
| D | A or G or T/U |
| H | A or C or T/U |
| V | A or C or G |
| N | any base |
| . or - | gap |

“PTC” refers to a premature termination codon. This is a stop codon introduced into a coding nucleic acid sequence by a nonsense mutation, i.e. a mutation in which a sense codon coding for one of the twenty proteinogenic amino acids specified by the standard genetic code is changed to a chain-terminating codon. The term thus refers to a premature stop signal in the translation of the genetic code contained in mRNA. The term “premature stop codon” (“PSC”) may be used synonymously for a premature termination codon, PTC.

The terms “frameshift suppression” or “frameshift rescue” refer to mechanisms masking the effects of a frameshift mutation and at least partly restoring the wild-type phenotype.

The terms “nonsense suppression”, “nonsense mutation suppression”, “PTC suppression” or “PTC rescue” refer to mechanisms masking the effects of a nonsense mutation and at least partly restoring mRNA translation and the wild-type phenotype.

The term “tRNA sequestration” relates to the irreversible binding of a tRNA to a tRNA synthetase such that the tRNA binds to the tRNA synthetase, but is not or essentially not released from the tRNA synthetase.

The term “suppressor tRNA” relates to a tRNA altering the reading of a messenger RNA in a given translation system such that, for example, a frameshift or nonsense mutation is “suppressed” and the effects of the frameshift or nonsense mutation are masked and the wild-type phenotype at least partly restored. An example of a suppressor tRNA is a tRNA carrying an amino acid and being able to base-pair to a PTC in an mRNA or, in case of a tRNA with an extended anticodon loop and a four-, five- or six-base anticodon, for example, to a section on an mRNA having two consecutive codons, one or both of which being mutated, or having a first and consecutive second codon, of which one is intact and one has an insertion or deletion. The translation system can thus correct the reading frame in the case of frameshift mutation or read through a PTC in the case of non-sense mutation.

The term “decoding activity” in relation to a tRNA refers to the property or capacity of a transfer RNA to be used by the translation machinery of a cell as an amino acid donor for the production of a protein. A tRNA has a “decoding activity” if, under physiologic conditions, it is charged with a cognate amino acid and the charged tRNA (aminoacyl-tRNA) is subsequently incorporated into a protein. The decoding activity of a first tRNA for a given amino acid can, for example, be compared with the decoding activity of a second tRNA for the same amino acid by comparing the proportion at which the amino acid of the first or second tRNA is incorporated in the protein. The terms “rescue activity”, “suppression activity”, “suppression efficiency” or “readthrough activity” refers to the decoding activity or efficiency of a suppressor tRNA, e.g. a frameshift suppressor or nonsense suppressor tRNA, in particular a tRNA designed to decode a premature stop codon in an mRNA into an amino acid, such that translation of the mRNA into the corresponding protein is not prematurely interrupted.

A term according to which a tRNA encoded by the DNA construct of the invention “still serves its function in the translational machinery of a living cell” relates to a tRNA transcribed from the tRNA gene having a decoding activity. In case of a suppressor tRNA, the term relates to a tRNA having a rescue activity greater than the spontaneous (stochastic) rescue activity in a given cellular environment.

The term “aminoacylation” relates to the enzymatic reaction in which a tRNA is charged with an amino acid. An aminoacyl tRNA synthetase (aaRS) catalyses the esterification of a specific cognate amino acid or its precursor to a compatible cognate tRNA to form an aminoacyl-tRNA. The term “aminoacyl-tRNA” thus relates to a tRNA with an amino acid attached to it. Each aminoacyl-tRNA synthetase is highly specific for a given amino acid, and, although more than one tRNA may be present for the same amino acid, there is only one aminoacyl-tRNA synthetase for each of the 20 proteinogenic amino acids. The terms “charge” or “load” may also be used synonymously for “aminoacylate”.

The term “expression” refers to the conversion of a genetic information into a functional product, for example the formation of a protein or a nucleic acid, e.g. functional RNA (e.g., transfer RNA), on the basis of the genetic information. The term not only encompasses the biosynthesis of a protein, e.g., an enzyme, based on genetic information including previous processes such as transcription or splicing, i.e., the formation of mRNA based on a DNA template, but also the synthesis of a functional RNA molecule, for example a tRNA. In relation to the production of a mature tRNA in a cell from a tRNA gene, in particular in terms of increased or decreased production, the term “transcription” may be used synonymously to the term “expression”, thus not only encompassing the synthesis of the first RNA product (i.e., the pre-tRNA) but also the processing of the first RNA product to the final RNA product (i.e., the mature tRNA). The terms “expression strength” or “expression level” relate to the amount of product synthesized from a DNA template. The term “high expression” relates to a high expression level, i.e. an expression level higher than an average expression level observed for a given molecule species, e.g., tRNA. The term “low expression” relates to a low expression level, i.e. an expression level lower than an average expression level observed for a given molecule species, e.g., tRNA.

The tRNA encoded by the nucleic acid, for example a suppressor tRNA, is arranged in the synthetic DNA construct, e.g., a synthetic vector, in a transcribable form, i.e. in a form that it is transcribed under natural conditions once introduced in a living mammalian cell, e.g., human cell, such that the encoded tRNA is produced in the cell, preferably using the natural transcription machinery of the cell. The tRNA may be arranged in a manner that it can be conditionally transcribed, i.e., depending on specific conditions in the cell. The sequence motifs in the 5′ leader sequence cause the tRNA arranged downstream from the 5′ leader sequence to be more strongly (at a higher level) or more weakly (at a lower level) transcribed than in the absence of these motifs.

The synthetic DNA construct according to the invention comprises at least one of the 5′ leader sequence motifs of features a), b) or c), or a combination of at least two sequence motifs independently selected from the sequence motifs of features a), b) or c). In a combination of sequence motifs, all sequence motifs can exclusively be chosen from the sequence motifs of one of the features a), b) or c), e.g. from the sequence motifs of feature b). Alternatively, in a combination of sequence motifs, a first sequence motif can be chosen from the sequence motifs of feature a) or b), whereas a second sequence motif is chosen from the sequence motifs of feature b). For example, the synthetic DNA construct according to the invention can comprise a first sequence motif selected from the sequence motifs of feature a) above, i.e. from the sequence motifs I_{L}, II_{L}, III_{L}, I_{H}, II_{H}, and III_{H}, and a second sequence motif selected from the sequence motifs of feature b). The synthetic DNA construct according to the invention can also comprise a combination of more than two, for example, three sequence motifs independently selected from the sequence motifs of features a), b) or c). The skilled person can, for example, combine the 5′ leader sequence motifs mentioned above, possibly in further combination with specific A and/or B boxes included in D or T arm sequences, to establish better control of expression of the tRNA encoded by the nucleic acid. The DNA construct of the invention does not encompass any naturally occurring combination of one or more 5′ leader sequence motifs with an encoded tRNA.

The invention provides, in a first embodiment, a DNA construct comprising, functionally linked to the nucleic acid encoding the transfer RNA, three sequence motifs, designated I_{H} (TGACCTAAGTGTAAAGT, SEQ ID NO: 1), II_{H} (TGAGATTTCCTTCAGGTT, SEQ ID NO: 2) and III_{H} (TATATAGTTCTGTATGAGACCACTCTTTCCC, SEQ ID NO: 3), promoting high expression of the tRNA, and three sequence motifs, I_{L} (ACCATAAACGTGAAATG, SEQ ID NO: 4), II_{L} (TCTTTGGATTTGGGAATC, SEQ ID NO: 5) and III_{L} (TTATAAGTTCTGTATGAGACCACTCTTTCCC, SEQ ID NO: 6), promoting low expression of the tRNA, when present in the 5′ leader sequence of the tRNA encoded in the DNA construct. The sequence motifs can be included in a 5′ leader sequence of an associated tRNA gene in order to control the level of expression of the tRNA.

In preferred embodiments of this first embodiment of a DNA construct of the invention, the sequence motifs I_{H} and I_{L} are not both present in the same 5′ leader sequence. The same applies to the sequence motifs II_{H} and II_{L} and to the sequence motifs III_{H} and III_{L}. Therefore, in a preferred embodiment of the synthetic DNA construct of the invention,
the 5′ leader sequence does not comprise the sequence motif I_{H} if it comprises the sequence motif I_{L}, and vice versa,
ii. the 5′ leader sequence does not comprise the sequence motif II_{H} if it comprises the sequence motif II_{L}, and vice versa, and
iii. the 5′ leader sequence does not comprise the sequence motif III_{H} if it comprises the sequence motif III_{L}, and vice versa.

In a preferred embodiment of the first embodiment of the synthetic DNA construct of the invention, the sequence motifs I_{L}, II_{L}, III_{L}, I_{H}, II_{H} or III_{H}, if present, are arranged upstream of the tRNA encoding nucleic acid at specific positions. In particular, the sequence motifs I_{L} and I_{H} are, if present, arranged in such a manner that they occupy positions −66 to −50, the positions relating to the region upstream of the nucleic acid encoding the tRNA. The sequence motifs II_{L} and II_{H} are, if present, arranged in such a manner that they occupy positions −49 to −32, and the sequence motifs III_{L} and III_{H} are, if present, arranged in such a manner that they occupy positions −31 to −1. In a preferred embodiment of the synthetic DNA construct according to the invention, the sequence motifs are thus preferably arranged such that
i. if the 5′ leader sequence comprises the sequence motif I_{H} or I_{L}, the sequence motif I_{H} or I_{L} has, in 5′-3′ direction, the positions-66 to −50,
ii. if the 5′ leader sequence comprises the sequence motif II_{H} or II_{L}, the sequence motif II_{H} or II_{L} has, in 5′-3′ direction, the positions −49 to −32, and
iii. if the 5′ leader sequence comprises the sequence motif III_{H} or III_{L}, the sequence motif III_{H} or III_{L} has, in 5′-3′ direction, the positions −31 to −1.

In a preferred embodiment of the first embodiment of the synthetic DNA construct of the invention, the 5′ leader sequence comprises at least two sequence motifs selected from the sequence motifs I_{L}, II_{L}, III_{L}, I_{H}, II_{H} or III_{H}, wherein the at least two sequence motifs are arranged, in 5′-3′ direction, in the order I_{H}-II_{H}, II_{H}-III_{H}, I_{H}-III_{H}, I_{L}-II_{L}, II_{L}-III_{L}, I_{L}-III_{L}, I_{H}-II_{L}, I_{H}-III_{L}, II_{H}-III_{L}, I_{L}-II_{H}, II_{L}-III_{H}, or I_{L}-III_{H}.

As mentioned above, the sequence motifs are preferably arranged such that they take the positions mentioned, i.e., positions-66 to −50 for sequence motifs I_{L} and I_{H}, positions-49 to −32 for sequence motifs II_{L} and II_{H}, and positions-31 to −1 for sequence motifs III_{L} and III_{H}.

In a further preferred embodiment of the synthetic DNA construct according to the invention, the 5′ leader sequence comprises three sequence motifs selected from the sequence motifs I_{L}, II_{L}, III_{L}, I_{H}, II_{H} or III_{H}. Preferably, the sequence motifs are arranged in the following order, in 5′-3′ direction: I_{L}-II_{L}-III_{L}, I_{H}-II_{H}-III_{H}, I_{H}-II_{L}-III_{L}, I_{L}-II_{H}-III_{L}, I_{L}-II_{L}-III_{H}, I_{H}-II_{H}-III_{L}, I_{H}-II_{L}-III_{H}, or I_{L}-II_{H}-III_{H}. Preferably, the sequence motifs are arranged directly one after the other, i.e., without nucleotides or linkers in between. Again, the sequence motifs preferably have the positions mentioned above.

In preferred embodiments of the first embodiment of the synthetic DNA construct of the invention, the 5′ leader sequence may have one of the sequences according to SEQ ID NOs: 12-37, according to the following list:

P1 (I_{H}-II_{H}-III_{H}, SEQ ID NO: 12):
TGACCTAAGTGTAAAGTTGAGATTTCCTTCAGGTTTATATAGTTCT
GTATGAGACCACTCTTTCCC
P2 (I_{L}-II_{L}-III_{L}, SEQ ID NO: 13):
ACCATAAACGTGAAATGTCTTTGGATTTGGGAATCTTATAAGTTCT
GTATGAGACCACTCTTTCCC
P3 (I_{H}, SEQ ID NO: 14):
TGACCTAAGTGTAAAGTNNNNNNNNNNNNNNNNNNNNNNNNNNNNN
NNNNNNNNNNNNNNNNNNNN
P4 (II_{H}, SEQ ID NO: 15):
NNNNNNNNNNNNNNNNNTGAGATTTCCTTCAGGTTNNNNNNNNNNN
NNNNNNNNNNNNNNNNNNNN
P5 (III_{H}, SEQ ID NO: 16):
NNNNNNNNNNNNNNNNNNNNNNNNNNNNNNNNNNNTATATAGTTCT
GTATGAGACCACTCTTTCCC
P6 (I_{H}-II_{H}, SEQ ID NO: 17):
TGACCTAAGTGTAAAGTTGAGATTTCCTTCAGGTTNNNNNNNNNNN
NNNNNNNNNNNNNNNNNNNN
P7 (II_{H}-III_{H}, SEQ ID NO: 18):
NNNNNNNNNNNNNNNNNTGAGATTTCCTTCAGGTTTATATAGTTCT
GTATGAGACCACTCTTTCCC
P8 (I_{H}-III_{H}, SEQ ID NO: 19):
TGACCTAAGTGTAAAGTNNNNNNNNNNNNNNNNNNTATATAGTTCT
GTATGAGACCACTCTTTCCC
P9 (I_{L}, SEQ ID NO: 20):
ACCATAAACGTGAAATGNNNNNNNNNNNNNNNNNNNNNNNNNNNNN
NNNNNNNNNNNNNNNNNNNN
P10 (II_{L}, SEQ ID NO: 21):
NNNNNNNNNNNNNNNNNTCTTTGGATTTGGGAATCNNNNNNNNNNN
NNNNNNNNNNNNNNNNNNNN
P11 (III_{L}, SEQ ID NO: 22):
NNNNNNNNNNNNNNNNNNNNNNNNNNNNNNNNNNNTTATAAGTTCT
GTATGAGACCACTCTTTCCC
P12 (I_{L}-II_{L}, SEQ ID NO: 23):
ACCATAAACGTGAAATGTCTTTGGATTTGGGAATCNNNNNNNNNNN
NNNNNNNNNNNNNNNNNNNN
P13 (II_{L}-III_{L}, SEQ ID NO: 24):
NNNNNNNNNNNNNNNNNTCTTTGGATTTGGGAATCTTATAAGTTCT
GTATGAGACCACTCTTTCCC
P14 (SEQ ID NO: 25):
ACCATAAACGTGAAATGNNNNNNNNNNNNNNNNNNTTATAAGTTCT
GTATGAGACCACTCTTTCCC
P15 (SEQ ID NO: 26):
TGACCTAAGTGTAAAGTTCTTTGGATTTGGGAATCNNNNNNNNNNN
NNNNNNNNNNNNNNNNNNNN
P16 (SEQ ID NO: 27):
TGACCTAAGTGTAAAGTNNNNNNNNNNNNNNNNNNTTATAAGTTCT
GTATGAGACCACTCTTTCCC
P17 (SEQ ID NO: 28):
NNNNNNNNNNNNNNNNNTGAGATTTCCTTCAGGTTTTATAAGTTCT
GTATGAGACCACTCTTTCCC
P18 (SEQ ID NO: 29):
ACCATAAACGTGAAATGTGAGATTTCCTTCAGGTTNNNNNNNNNNN
NNNNNNNNNNNNNNNNNNNN
P19 (SEQ ID NO: 30):
NNNNNNNNNNNNNNNNNTCTTTGGATTTGGGAATCTATATAGTTCT
GTATGAGACCACTCTTTCCC
P20 (SEQ ID NO: 31):
ACCATAAACGTGAAATGNNNNNNNNNNNNNNNNNNTATATAGTTCT
GTATGAGACCACTCTTTCCC
P21 (SEQ ID NO: 32):
TGACCTAAGTGTAAAGTTCTTTGGATTTGGGAATCTTATAAGTTCT
GTATGAGACCACTCTTTCCC
P22 (SEQ ID NO: 33):
ACCATAAACGTGAAATGTGAGATTTCCTTCAGGTTTTATAAGTTCT
GTATGAGACCACTCTTTCCC
P23 (SEQ ID NO: 34):
ACCATAAACGTGAAATGTCTTTGGATTTGGGAATCTATATAGTTCT
GTATGAGACCACTCTTTCCC
P24 (SEQ ID NO: 35):
TGACCTAAGTGTAAAGTTGAGATTTCCTTCAGGTTTTATAAGTTCT
GTATGAGACCACTCTTTCCC
P25 (SEQ ID NO: 36):
TGACCTAAGTGTAAAGTTCTTTGGATTTGGGAATCTATATAGTTCT
GTATGAGACCACTCTTTCCC
P26 (SEQ ID NO: 37):
ACCATAAACGTGAAATGTGAGATTTCCTTCAGGTTTATATAGTTCT
GTATGAGACCACTCTTTCCC
N stands for any nucleotide (A, G, C or T).

In a preferred embodiment of the first embodiment of the synthetic DNA construct of the invention, the 5′ leader sequence described above is flanked, at its 3′ end, i.e. in the direction downstream to the tRNA gene, or at its 5′ end, i.e. in the direction upstream from the tRNA gene and the 5′ leader sequence described above, by a linker or spacer region comprising repeats of a linker/spacer sequence. The terms “linker” and “spacer” are used synonymously in this context. The spacer region can, for example, comprise a nucleotide sequence of up to 300 nt in length. The spacer region preferably comprises or consists of consecutive repeats, for example 10-30 consecutive repeats, of a spacer sequence of 8-12, 9-11 or 10 nucleotides. An example of a suitable spacer sequence is ACTCTTTCCC (SEQ ID NO: 38). A 5′ leader sequence according to the first embodiment of the synthetic DNA construct of the invention including such a spacer region can, for example, have the following general structure: I_{H}-II_{H}-III_{H}-(spacer)ₙ, or (spacer)ₙ-I_{H}-II_{H}-III_{H} with n=10-30. The number and composition of the sequence motifs can vary, although it is preferred to have a combination of at least two or three different sequence motifs selected from the sequence motifs I_{H}, II_{H} III_{H}, I_{L}, II_{L}, and III_{L}, plus the spacer region at the 5′ end or 3′ end of the combination. The term “sequence motif region” is used for the region with the at least one sequence motif or the combination of sequence motifs. The spacer region can be directly adjacent to the 5′ end or 3′ end of the sequence motif region, or can be separated, in 5′ or 3′ direction, from the sequence motif region by a number of nucleotides, preferably not more than 50, 40, 30, 25, 20, 15 or 10 nucleotides. In an embodiment in which the 5′ leader sequence comprises spacer repeats, the positions mentioned above are preferably adapted accordingly in that the 5′ leader sequence extends in 5′ direction by the number of nucleotides covered by the spacer region.

In a second embodiment of the synthetic DNA construct of the invention, the 5′ leader sequence comprises one sequence motif selected from the group consisting of the sequence motifs

(SEQ ID NO: 7)
VNANANTVHANANNTTNNNATRANATTCNGDGVGNAANATAB
TNCTVGND,
designated 50 nt_{H},
and
(SEQ ID NO: 8)
GCNGGDGGCGNGTTCBBCTGTNVNAGCNTNCGDNGNCGTTTN
NCNTCGGT,
designated 50 nt_{L}.

The abbreviations have the standard meaning as indicated above (see, e.g., ST.26 ver. 1.5).

The sequence motif 50 nt_{H} promotes a higher expression of the downstream tRNA gene, and 50 nt_{L} causes a lower expression of the downstream tRNA gene.

In a preferred embodiment of this second embodiment of the synthetic DNA construct of the invention the 5′ leader sequence does not comprise the sequence motif 50 nt_{H} if it comprises the sequence motif 50 nt_{L}, and vice versa.

Further preferred, in this second embodiment, the sequence motifs 50 nt_{H} or 50 nt have the positions −50 to −1, i.e., if the sequence of 50 nt_{H} is present, it preferably takes, in 5′-3′ direction, the positions −50 to −1, and if the sequence of 50 nt_{L} is present, it takes, in 5′-3′ direction, positions-50 to −1.

The sequence motif 50 nt_{H} may have one of the following sequences:

P27
(50nt_{H1}, SEQ ID NO: 39)
CCATGATCCCCCACTATTAAGGATATCCGGAGAGGATGCTACCTATCAGG
P28
(50nt_{H2}, SEQ ID NO: 40)
AGACCAGCTGTATAGCCTCAGAATGATCCGTCCGGAAACCCTTACTAGGT
P29
(50nt_{H3}, SEQ ID NO: 41)
GCATATTGCAGAACTTGTGAAGAGGCTTTATGCGCCACACACTGCAAGCA
P30
(50nt_{H4}, SEQ ID NO: 42)
GCAAAAGCAAAAGCATTAAGTAGCTTTCTGGCATAAACATATTGCAGCTG
P31
(50nt_{H5}, SEQ ID NO: 43)
GGTTGCGGTAAGCATTAGAGGGCTATCAGCAGCATCTTATCGCAGCGGAG
P32 50nt_{H6},
(SEQ ID NO: 44)
CTGCAGTATAACCTTGAAGTACCATCACGGAGAGAAACAGTGCCATCTCA
P33
(50nt_{H7}, SEQ ID NO: 45)
AACGACTACGTAGTGTTCTATAACAATCATGAGAAATTTTAGTTCTAGAT

The sequence motif 50 nt_{L} may have one of the following sequences:

P34
(50nt_{L1}, SEQ ID NO: 46)
GCTGGTGGCGAGTTCCGCTGTGCCAGCTTCCGTTGGCGTTTGCCATCGGT
P35
(50nt_{L4}, SEQ ID NO: 47)
ATGCGGCCTTGTGTGGTGGCTCATTGTGGTGGGCCAGAAAACGCGTGCAA
P36
(50nt_{L5}, SEQ ID NO: 48)
GCCAGAGGCGCTGGGGCCAGGAGGCGCAAGCCGGGCCCTGAAGCCGCCTC
P37
(50nt_{L6}, SEQ ID NO: 49)
AAAGAAATTAGGAAATTCCTGTGGAAGCTGGCGCGTTGATGCACTTCGTC
P38
(50nt_{L7}, SEQ ID NO: 50)
CAACAAACATTTTGCTTTTTTAAAATTGAAAGAACAACTGTTTTCCGGGT

In a preferred embodiment of this second embodiment of the synthetic DNA construct of the invention, the 5′ leader sequence comprises one of the sequence motifs of SEQ ID NO: 7 or 8 of feature b) above, with the proviso that the 5′ leader sequence does not comprise one of the above sequences of SEQ ID NO: 39, 42 or 46; or, if the 5′ leader sequence comprises one of the sequences of SEQ ID NO: 39 or 46, the encoded transfer RNA is not a tRNA^{Gly}, and, if the 5′ leader sequence comprises the sequence of SEQ ID NO: 42, the encoded transfer RNA is not a tRNA^{Leu}. This is, however, preferably only the case for a synthetic DNA construct in which a sequence motif of SEQ ID NO: 7 or 8 of feature b) is not combined with one of the sequence motifs of feature a) or c) above (see below). The term “if the 5′ leader sequence comprises one of the sequences of SEQ ID NO: 39 or 46, the encoded transfer RNA is not a tRNA^{Gly}” means that the nucleic acid of a synthetic DNA construct of the invention comprising one of the sequences of SEQ ID NO: 39 or 46 as a 5′ leader sequence does not encode a transfer RNA having a tRNA body that is aminoacylated, in vivo, with glycine. In the same manner, the term “if the 5′ leader sequence comprises the sequence of SEQ ID NO: 42, the encoded transfer RNA is not a tRNA^{Leu}” means that the nucleic acid of a synthetic DNA construct of the invention comprising the sequence of SEQ ID NO: 42 as a 5′ leader sequence does not encode a transfer RNA having a tRNA body that is aminoacylated, in vivo, with leucine.

In a further preferred embodiment of the synthetic DNA construct according to the invention, the 5′ leader sequence comprises one of the sequence motifs selected from the sequence motifs I_{L}, II_{L}, III_{L}, I_{H}, II_{H} and III_{H} of feature a) above, possibly including a spacer region as described above, followed immediately, in 5′-3′ direction, by one of the sequence motifs selected from the sequence motifs 50 nt_{H} and 50 nt_{L} of feature b) above. In this embodiment, one of the sequence motifs I_{H} (SEQ ID NO: 1), II_{H} (SEQ ID NO: 2), III_{H} (SEQ ID NO: 3), I_{L} (SEQ ID NO: 4), II_{L} (SEQ ID NO: 5) and III_{L} (SEQ ID NO: 6) of feature a) above is combined with one of the sequence motifs 50 nt_{H} (SEQ ID NO: 7) or 50 nt_{L} (SEQ ID NO: 8) of feature b) above. Preferably, in this embodiment, the sequence motif 50 nt_{H} or 50 nt_{L} immediately follows the sequence motif I_{H}, II_{H}, III_{H}, I_{L}, II_{L} or III_{L}. Any of the combinations is possible. It is thus possible to combine, for example, the sequence motif II_{L} with the sequence motif 50 nt_{L}, the sequence motif II_{L}, with the sequence motif 50 nt_{H}, or the sequence motif I_{H} with the sequence motif 50 nt_{L} or 50 nt_{H}. The following combinations are thus possible: I_{H}-50 nt_{H}; I_{H}-50 nt_{L}; II_{H}-50 nt_{H}; II_{H}-50 nt_{L}; III_{H}-50 nt_{H}; III_{H}-50 nt_{L}; I_{L}-50 nt_{H}; I_{L}-50 nt_{L}; II_{L}-50 nt_{H}; II_{L}-50 nt_{L}; III_{L}-50 nt_{H}; III_{L}-50 nt_{L}. In this embodiment, the 5′ leader sequence thus comprises or has the following sequence according to SEQ ID NO: 50-61:

I_{H} - 50nt_{H}
(SEQ ID NO: 51)
TGACCTAAGTGTAAAGTVNANANTVHANANNTTNNNATRANATTCNGDGVGNAAN
ATABTNCTVGND
I_{H} - 50nt_{L}
(SEQ ID NO: 52)
TGACCTAAGTGTAAAGTGCNGGDGGCGNGTTCBBCTGTNVNAGCNTNCGDNGNCG
TTTNNCNTCGGT
II_{H} - 50nt_{H}
(SEQ ID NO: 53)
TGAGATTTCCTTCAGGTTVNANANTVHANANNTTNNNATRANATTCNGDGVGNAA
NATABTNCTVGND
II_{H} - 50nt_{L}
(SEQ ID NO: 54)
TGAGATTTCCTTCAGGTTGCNGGDGGCGNGTTCBBCTGTNVNAGCNTNCGDNGNCG
TTTNNCNTCGGT
III_{H} - 50nt_{H}
(SEQ ID NO: 55)
TATATAGTTCTGTATGAGACCACTCTTTCCCVNANANTVHANANNTTNNNATRANA
TTCNGDGVGNAANATABTNCTVGND
III_{H} - 50nt_{L}
(SEQ ID NO: 56)
TATATAGTTCTGTATGAGACCACTCTTTCCCGCNGGDGGCGNGTTCBBCTGTNVNA
GCNTNCGDNGNCGTTTNNCNTCGGT
I_{L} - 50nt_{H}
(SEQ ID NO: 57)
ACCATAAACGTGAAATGVNANANTVHANANNTTNNNATRANATTCNGDGVGNAAN
ATABTNCTVGND
I_{L} - 50nt_{L}
(SEQ ID NO: 58)
ACCATAAACGTGAAATGGCNGGDGGCGNGTTCBBCTGTNVNAGCNTNCGDNGNCG
TTTNNCNTCGGT
II_{L} - 50nt_{H}
(SEQ ID NO: 59)
TCTTTGGATTTGGGAATCVNANANTVHANANNTTNNNATRANATTCNGDGVGNAA
NATABTNCTVGND
II_{L} - 50nt_{L}
(SEQ ID NO: 60)
TCTTTGGATTTGGGAATCGCNGGDGGCGNGTTCBBCTGTNVNAGCNTNCGDNGNCG
TTTNNCNTCGGT
III_{L} - 50nt_{H}
(SEQ ID NO: 61)
TTATAAGTTCTGTATGAGACCACTCTTTCCCVNANANTVHANANNTTNNNATRANA
TTCNGDGVGNAANATABTNCTVGND
III_{L} - 50nt_{L}
(SEQ ID NO: 62)
TTATAAGTTCTGTATGAGACCACTCTTTCCCGCNGGDGGCGNGTTCBBCTGTNVNA
GCNTNCGDNGNCGTTTNNCNTCGGT

Considering the sequences of the 50 nt_{H} and 50 nt_{L} motifs above (P27 to P38) the 5′ leader sequence can comprise or have one of the following sequences:

(SEQ ID NO: 63)
TGACCTAAGTGTAAAGTCCATGATCCCCCACTATTAAGGATATCCGGAGAGGATGC
TACCTATCAGG
(SEQ ID NO: 64)
TGACCTAAGTGTAAAGTAGACCAGCTGTATAGCCTCAGAATGATCCGTCCGGAAAC
CCTTACTAGGT
(SEQ ID NO: 65)
TGACCTAAGTGTAAAGTGCATATTGCAGAACTTGTGAAGAGGCTTTATGCGCCACA
CACTGCAAGCA
(SEQ ID NO: 66)
TGACCTAAGTGTAAAGTGCAAAAGCAAAAGCATTAAGTAGCTTTCTGGCATAAACA
TATTGCAGCTG
(SEQ ID NO: 67)
TGACCTAAGTGTAAAGTGGTTGCGGTAAGCATTAGAGGGCTATCAGCAGCATCTTA
TCGCAGCGGAG
(SEQ ID NO: 68)
TGACCTAAGTGTAAAGTCTGCAGTATAACCTTGAAGTACCATCACGGAGAGAAACA
GTGCCATCTCA
(SEQ ID NO: 69)
TGACCTAAGTGTAAAGTAACGACTACGTAGTGTTCTATAACAATCATGAGAAATTT
TAGTTCTAGAT
(SEQ ID NO: 70)
TGACCTAAGTGTAAAGTGCTGGTGGCGAGTTCCGCTGTGCCAGCTTCCGTTGGCGT
TTGCCATCGGT
(SEQ ID NO: 71)
TGACCTAAGTGTAAAGTATGCGGCCTTGTGTGGTGGCTCATTGTGGTGGGCCAGAA
AACGCGTGCAA
(SEQ ID NO: 72)
TGACCTAAGTGTAAAGTGCCAGAGGCGCTGGGGCCAGGAGGCGCAAGCCGGGCCC
TGAAGCCGCCTC
(SEQ ID NO: 73)
TGACCTAAGTGTAAAGTAAAGAAATTAGGAAATTCCTGTGGAAGCTGGCGCGTTGA
TGCACTTCGTC
(SEQ ID NO: 74)
TGACCTAAGTGTAAAGTCAACAAACATTTTGCTTTTTTAAAATTGAAAGAACAACT
GTTTTCCGGGT
(SEQ ID NO: 75)
TGAGATTTCCTTCAGGTTCCATGATCCCCCACTATTAAGGATATCCGGAGAGGATG
CTACCTATCAGG
(SEQ ID NO: 76)
TGAGATTTCCTTCAGGTTAGACCAGCTGTATAGCCTCAGAATGATCCGTCCGGAAA
CCCTTACTAGGT
(SEQ ID NO: 77)
TGAGATTTCCTTCAGGTTGCATATTGCAGAACTTGTGAAGAGGCTTTATGCGCCAC
ACACTGCAAGCA
(SEQ ID NO: 78)
TGAGATTTCCTTCAGGTTGCAAAAGCAAAAGCATTAAGTAGCTTTCTGGCATAAAC
ATATTGCAGCTG
(SEQ ID NO: 79)
TGAGATTTCCTTCAGGTTGGTTGCGGTAAGCATTAGAGGGCTATCAGCAGCATCTT
ATCGCAGCGGAG
(SEQ ID NO: 80)
TGAGATTTCCTTCAGGTTCTGCAGTATAACCTTGAAGTACCATCACGGAGAGAAAC
AGTGCCATCTCA
(SEQ ID NO: 81)
TGAGATTTCCTTCAGGTTAACGACTACGTAGTGTTCTATAACAATCATGAGAAATTT
TAGTTCTAGAT
(SEQ ID NO: 82)
TGAGATTTCCTTCAGGTTGCTGGTGGCGAGTTCCGCTGTGCCAGCTTCCGTTGGCGT
TTGCCATCGGT
(SEQ ID NO: 83)
TGAGATTTCCTTCAGGTTATGCGGCCTTGTGTGGTGGCTCATTGTGGTGGGCCAGAA
AACGCGTGCAA
(SEQ ID NO: 84)
TGAGATTTCCTTCAGGTTGCCAGAGGCGCTGGGGCCAGGAGGCGCAAGCCGGGCC
CTGAAGCCGCCTC
(SEQ ID NO: 85)
TGAGATTTCCTTCAGGTTAAAGAAATTAGGAAATTCCTGTGGAAGCTGGCGCGTTG
ATGCACTTCGTC
(SEQ ID NO: 86)
TGAGATTTCCTTCAGGTTCAACAAACATTTTGCTTTTTTAAAATTGAAAGAACAACT
GTTTTCCGGGT
(SEQ ID NO: 87)
TATATAGTTCTGTATGAGACCACTCTTTCCCCCATGATCCCCCACTATTAAGGATAT
CCGGAGAGGATGCTACCTATCAGG
(SEQ ID NO: 88)
TATATAGTTCTGTATGAGACCACTCTTTCCCAGACCAGCTGTATAGCCTCAGAATGA
TCCGTCCGGAAACCCTTACTAGGT
(SEQ ID NO: 89)
TATATAGTTCTGTATGAGACCACTCTTTCCCGCATATTGCAGAACTTGTGAAGAGGC
TTTATGCGCCACACACTGCAAGCA
(SEQ ID NO: 90)
TATATAGTTCTGTATGAGACCACTCTTTCCCGCAAAAGCAAAAGCATTAAGTAGCT
TTCTGGCATAAACATATTGCAGCTG
(SEQ ID NO: 91)
TATATAGTTCTGTATGAGACCACTCTTTCCCGGTTGCGGTAAGCATTAGAGGGCTAT
CAGCAGCATCTTATCGCAGCGGAG
(SEQ ID NO: 92)
TATATAGTTCTGTATGAGACCACTCTTTCCCCTGCAGTATAACCTTGAAGTACCATC
ACGGAGAGAAACAGTGCCATCTCA
(SEQ ID NO: 93)
TATATAGTTCTGTATGAGACCACTCTTTCCCAACGACTACGTAGTGTTCTATAACAA
TCATGAGAAATTTTAGTTCTAGAT
(SEQ ID NO: 94)
TATATAGTTCTGTATGAGACCACTCTTTCCCGCTGGTGGCGAGTTCCGCTGTGCCAG
CTTCCGTTGGCGTTTGCCATCGGT
(SEQ ID NO: 95)
TATATAGTTCTGTATGAGACCACTCTTTCCCATGCGGCCTTGTGTGGTGGCTCATTG
TGGTGGGCCAGAAAACGCGTGCAA
(SEQ ID NO: 96)
TATATAGTTCTGTATGAGACCACTCTTTCCCGCCAGAGGCGCTGGGGCCAGGAGGC
GCAAGCCGGGCCCTGAAGCCGCCTC
(SEQ ID NO: 97)
TATATAGTTCTGTATGAGACCACTCTTTCCCAAAGAAATTAGGAAATTCCTGTGGA
AGCTGGCGCGTTGATGCACTTCGTC
(SEQ ID NO: 98)
TATATAGTTCTGTATGAGACCACTCTTTCCCCAACAAACATTTTGCTTTTTTAAAAT
TGAAAGAACAACTGTTTTCCGGGT
(SEQ ID NO: 99)
ACCATAAACGTGAAATGCCATGATCCCCCACTATTAAGGATATCCGGAGAGGATGC
TACCTATCAGG
(SEQ ID NO: 100)
ACCATAAACGTGAAATGAGACCAGCTGTATAGCCTCAGAATGATCCGTCCGGAAA
CCCTTACTAGGT
(SEQ ID NO: 101)
ACCATAAACGTGAAATGGCATATTGCAGAACTTGTGAAGAGGCTTTATGCGCCACA
CACTGCAAGCA
(SEQ ID NO: 102)
ACCATAAACGTGAAATGGCAAAAGCAAAAGCATTAAGTAGCTTTCTGGCATAAAC
ATATTGCAGCTG
(SEQ ID NO: 103)
ACCATAAACGTGAAATGGGTTGCGGTAAGCATTAGAGGGCTATCAGCAGCATCTTA
TCGCAGCGGAG
(SEQ ID NO: 104)
ACCATAAACGTGAAATGCTGCAGTATAACCTTGAAGTACCATCACGGAGAGAAAC
AGTGCCATCTCA
(SEQ ID NO: 105)
ACCATAAACGTGAAATGAACGACTACGTAGTGTTCTATAACAATCATGAGAAATTT
TAGTTCTAGAT
(SEQ ID NO: 106)
ACCATAAACGTGAAATGGCTGGTGGCGAGTTCCGCTGTGCCAGCTTCCGTTGGCGT
TTGCCATCGGT
(SEQ ID NO: 107)
ACCATAAACGTGAAATGATGCGGCCTTGTGTGGTGGCTCATTGTGGTGGGCCAGAA
AACGCGTGCAA
(SEQ ID NO: 108)
ACCATAAACGTGAAATGGCCAGAGGCGCTGGGGCCAGGAGGCGCAAGCCGGGCCC
TGAAGCCGCCTC
(SEQ ID NO: 109)
ACCATAAACGTGAAATGAAAGAAATTAGGAAATTCCTGTGGAAGCTGGCGCGTTG
ATGCACTTCGTC
(SEQ ID NO: 110)
ACCATAAACGTGAAATGCAACAAACATTTTGCTTTTTTAAAATTGAAAGAACAACT
GTTTTCCGGGT
(SEQ ID NO: 111)
TCTTTGGATTTGGGAATCCCATGATCCCCCACTATTAAGGATATCCGGAGAGGATG
CTACCTATCAGG
(SEQ ID NO: 112)
TCTTTGGATTTGGGAATCAGACCAGCTGTATAGCCTCAGAATGATCCGTCCGGAAA
CCCTTACTAGGT
(SEQ ID NO: 113)
TCTTTGGATTTGGGAATCGCATATTGCAGAACTTGTGAAGAGGCTTTATGCGCCAC
ACACTGCAAGCA
(SEQ ID NO: 114)
TCTTTGGATTTGGGAATCGCAAAAGCAAAAGCATTAAGTAGCTTTCTGGCATAAAC
ATATTGCAGCTG
(SEQ ID NO: 115)
TCTTTGGATTTGGGAATCGGTTGCGGTAAGCATTAGAGGGCTATCAGCAGCATCTT
ATCGCAGCGGAG
(SEQ ID NO: 116)
TCTTTGGATTTGGGAATCCTGCAGTATAACCTTGAAGTACCATCACGGAGAGAAAC
AGTGCCATCTCA
(SEQ ID NO: 117)
TCTTTGGATTTGGGAATCAACGACTACGTAGTGTTCTATAACAATCATGAGAAATTT
TAGTTCTAGAT
(SEQ ID NO: 118)
TCTTTGGATTTGGGAATCGCTGGTGGCGAGTTCCGCTGTGCCAGCTTCCGTTGGCGT
TTGCCATCGGT
(SEQ ID NO: 119)
TCTTTGGATTTGGGAATCATGCGGCCTTGTGTGGTGGCTCATTGTGGTGGGCCAGA
AAACGCGTGCAA
(SEQ ID NO: 120)
TCTTTGGATTTGGGAATCGCCAGAGGCGCTGGGGCCAGGAGGCGCAAGCCGGGCC
CTGAAGCCGCCTC
(SEQ ID NO: 121)
TCTTTGGATTTGGGAATCAAAGAAATTAGGAAATTCCTGTGGAAGCTGGCGCGTTG
ATGCACTTCGTC
(SEQ ID NO: 122)
TCTTTGGATTTGGGAATCCAACAAACATTTTGCTTTTTTAAAATTGAAAGAACAACT
GTTTTCCGGGT
(SEQ ID NO: 123)
TTATAAGTTCTGTATGAGACCACTCTTTCCCCCATGATCCCCCACTATTAAGGATAT
CCGGAGAGGATGCTACCTATCAGG
(SEQ ID NO: 124)
TTATAAGTTCTGTATGAGACCACTCTTTCCCAGACCAGCTGTATAGCCTCAGAATGA
TCCGTCCGGAAACCCTTACTAGGT
(SEQ ID NO: 125)
TTATAAGTTCTGTATGAGACCACTCTTTCCCGCATATTGCAGAACTTGTGAAGAGGC
TTTATGCGCCACACACTGCAAGCA
(SEQ ID NO: 126)
TTATAAGTTCTGTATGAGACCACTCTTTCCCGCAAAAGCAAAAGCATTAAGTAGCT
TTCTGGCATAAACATATTGCAGCTG
(SEQ ID NO: 127)
TTATAAGTTCTGTATGAGACCACTCTTTCCCGGTTGCGGTAAGCATTAGAGGGCTAT
CAGCAGCATCTTATCGCAGCGGAG
(SEQ ID NO: 128)
TTATAAGTTCTGTATGAGACCACTCTTTCCCCTGCAGTATAACCTTGAAGTACCATC
ACGGAGAGAAACAGTGCCATCTCA
(SEQ ID NO: 129)
TTATAAGTTCTGTATGAGACCACTCTTTCCCAACGACTACGTAGTGTTCTATAACAA
TCATGAGAAATTTTAGTTCTAGAT
(SEQ ID NO: 130)
TTATAAGTTCTGTATGAGACCACTCTTTCCCGCTGGTGGCGAGTTCCGCTGTGCCAG
CTTCCGTTGGCGTTTGCCATCGGT
(SEQ ID NO: 131)
TTATAAGTTCTGTATGAGACCACTCTTTCCCATGCGGCCTTGTGTGGTGGCTCATTG
TGGTGGGCCAGAAAACGCGTGCAA
(SEQ ID NO: 132)
TTATAAGTTCTGTATGAGACCACTCTTTCCCGCCAGAGGCGCTGGGGCCAGGAGGC
GCAAGCCGGGCCCTGAAGCCGCCTC
(SEQ ID NO: 133)
TTATAAGTTCTGTATGAGACCACTCTTTCCCAAAGAAATTAGGAAATTCCTGTGGA
AGCTGGCGCGTTGATGCACTTCGTC
(SEQ ID NO: 134)
TTATAAGTTCTGTATGAGACCACTCTTTCCCCAACAAACATTTTGCTTTTTTAAAAT
TGAAAGAACAACTGTTTTCCGGGT

In further preferred embodiment of this embodiment of the synthetic DNA construct according to the invention, the 5′ leader sequence comprises one of the sequence motifs I_{H}, II_{H}, III_{H}, followed immediately, in 5′-3′ direction, by the sequence motif 50 nt_{H}; or comprises one of the sequence motifs I_{L}, II_{L}, III_{L}, followed immediately, in 5′-3′ direction, by the sequence motif 50 nt_{L}. A 5′ leader sequence comprising one of the sequence motifs I_{H}, II_{H}, III_{H}, followed immediately, in 5′-3′ direction, by the sequence motif 50 nt_{H} can comprise or have one of the sequences of SEQ ID NO: 51, 53 or 55, or, for example, one of the sequences of SEQ ID NO: 63-69, 75-81, 87-93. A 5′ leader sequence comprising one of the sequence motifs I_{L}, II_{L}, III_{L}, followed immediately, in 5′-3′ direction, by the sequence motif 50 nt_{L} can comprise or have one of the sequences of SEQ ID NO: 58, 60 or 62, or, for example, one of the sequences of SEQ ID NO: 106-110, 118-122, 130-134. As mentioned above, however, it is also possible to combine any one of the lower expression motifs (denoted with the subscript “L”) with any higher expression motif (denoted with the subscript “H”).

In a preferred embodiment of the second embodiment of the synthetic DNA construct according to the invention, the 5′ leader sequence comprises i) one of the sequence motifs selected from the sequence motifs I_{H}, II_{H} III_{H}, I_{L}, II_{L}, and III_{L} of feature a) above, followed immediately, in 5′-3′ direction, by a spacer region comprising or composed of 10-30 repeats of a spacer sequence of 8-12, preferably 9-11, more preferably 10 nucleotides, followed immediately, in 5′-3′ direction, by one of the sequence motifs selected from the sequence motifs 50 nt_{L} and 50 nt_{H} of feature b) above, or ii) one of the sequence motifs selected from the sequence motifs I_{H}, II_{H} III_{H}, I_{L}, II_{L}, and III_{L} of feature a) above, flanked, at its 5′ end, by a spacer region comprising or composed of 10-30 repeats of a spacer sequence of 8-12, preferably 9-11, more preferably 10 nucleotides, and at its 3′ end, by one of the sequence motifs selected from the sequence motifs 50 nt_{L} and 50 nt_{H} of feature b) above. The spacer sequence can, for example have the sequence ACTCTTTCCC (SEQ ID NO: 38). The term “first sequence motif region” may used for the region comprising or composed of the sequence motifs selected from the sequence motifs I_{H}, II_{H} III_{H}, I_{L}, II_{L}, and III_{L}, and the term “second sequence motif region” may be used for the region comprising or composed of the sequence motifs selected from the sequence motifs 50 nt_{L} and 50 nt_{H}. As mentioned above for the first embodiment, the number and composition of the sequence motifs of the first sequence motif region can vary, although it is preferred to have a combination of at least two or three different sequence motifs selected from the sequence motifs I_{H}, II_{H} III_{H}, I_{L}, II_{L}, and III_{L}, plus the spacer region at the 5′ end or 3′ end of the first sequence motif region, plus a sequence motif selected from the sequence motifs 50 nt_{L} and 50 nt_{H} of feature b) above, at the 3′ end of the combination. A spacer region at the 5′ end of the first sequence motif region can be directly adjacent to the 5′ end of the first sequence motif region, or can be separated, in 5′ direction, from the first sequence motif region by a number of nucleotides, preferably not more than 50, 40, 30, 25, 20, 15 or 10 nucleotides.

In the synthetic DNA construct of the invention, it is also possible to combine any two or three of the sequence motifs I_{L}, II_{L}, III_{L}, I_{H}, II_{H}, III_{H} arranged in a row, including a spacer region as describe above or not at the 5′ end or 3′ end, with one of the sequence motifs 50 nt_{H} or 50 nt_{L}.

In a preferred embodiment of the synthetic DNA construct of the invention, the 5′ leader sequence and the encoded transfer RNA are not from the same tRNA, if the tRNA is a naturally occurring tRNA, i.e, if the nucleic acid of the synthetic DNA construct of the invention encodes a naturally occurring transfer RNA, the 5′ leader sequence is not from said naturally occurring transfer RNA.

In a third embodiment of the synthetic DNA construct of the invention, the 5′ leader sequence of the nucleic acid encoding a transfer RNA comprises at least one sequence motif selected from the sequence motifs GAAATGCCTT, designated 10n_{H1}, (SEQ ID NO: 9), GTGGGAACTA, designated 10 nt_{H2}, (SEQ ID NO: 10), and GTGTTGCTTG, designated 10 nt_{H3}, (SEQ ID NO: 11). The sequence motifs, designated 10 nt_{H1}, 10 nt_{H2} and 10 nt_{H3}, can advantageously be used to promote higher expression of the tRNA gene arranged downstream of the 5′ leader sequence within the synthetic DNA construct. The motifs are preferably arranged within a region from −100 to −1 in relation to the tRNA gene.

Preferably, the 5′ leader sequence, in this third embodiment of a synthetic DNA construct of the invention, comprises at least two sequence motifs selected from the sequence motifs 10 nt_{H1}, 10 nt_{H2} and 10 nt_{H3}. Preferably, the at least two sequence motifs are:
i. GAAATGCCTT, 10 nt_{H1}, (SEQ ID NO: 9) and GTGTTGCTTG, 10 nt_{H3}, (SEQ ID NO: 11), wherein the sequence motifs are arranged, in 5′-3′ direction, in the following order: GTGTTGCTTG (SEQ ID NO: 11)-GAAATGCCTT (SEQ ID NO: 9), i.e., 10 nt_{H3}-10 nt_{H1}, or
ii. GAAATGCCTT, 10 nt_{H1}, (SEQ ID NO: 9) and GTGGGAACTA, 10 nt_{H2}, (SEQ ID NO: 10), wherein the sequence motifs are arranged, in 5′-3′ direction, in the following order: GAAATGCCTT (SEQ ID NO: 9)-GTGGGAACTA (SEQ ID NO: 10), i.e., 10 nt_{H1}-10 nt_{H2}, or
iii. GTGGGAACTA, 10 nt_{H2}, (SEQ ID NO: 10), and GTGTTGCTTG, 10 nt_{H3}, (SEQ ID NO: 11), wherein the sequence motifs are arranged, in 5′-3′ direction, in the following order: GTGTTGCTTG (SEQ ID NO: 11)-GTGGGAACTA (SEQ ID NO: 10), i.e., 10 nt_{H3}-10 nt_{H2}.

Further preferred, in this third embodiment of a synthetic DNA construct of the invention, the 5′ leader sequence comprises all three sequence motifs GAAATGCCTT (SEQ ID NO: 9), 10 nt_{H1}, GTGGGAACTA (SEQ ID NO: 10), 10 nt_{H2}, and GTGTTGCTTG (SEQ ID NO: 11), 10 nt_{H3}, preferably in the following order, in 5′-3′ direction: GTGTTGCTTG (SEQ ID NO: 11)-GAAATGCCTT (SEQ ID NO: 9)-GTGGGAACTA (SEQ ID NO: 10), i.e., 10 nt_{H3}-10 nt_{H1}-10 nt_{H2}.

The tRNA encoded by the nucleic acid may be a naturally occurring tRNA, or an engineered, for example microbiologically engineered, tRNA. Preferably the tRNA is engineered, for example, in that the tRNA has an altered anticodon, e.g., an anticodon base-pairing to a stop codon in an mRNA, or has an extended anticodon loop comprising, e.g., a four-, five-, or six-base anticodon, or has a modified D arm, Anticodon arm, T arm or acceptor stem.

In a preferred embodiment of the synthetic DNA construct according to the invention, the nucleic acid encoding the tRNA comprises a B box comprising the sequence H₅₄T₅₅C₅₆G₅₇A₅₈N₅₉T₆₀, preferably, T₅₄T₅₅C₅₆G₅₇A₅₈N₅₉T₆₀, the index representing the positions of the nucleotides in the transfer RNA, the position numbering following transfer RNA numbering convention. N stands for any nucleotide (A, C, G or T), H stands for A, C, or T. Nucleotides with number 54-60 are, in the mature tRNA, arranged within the T arm of the mature tRNA, and form the T loop. A tRNA comprising a B box having the above sequence is transcribed more strongly than a tRNA lacking such a B box. The encoded tRNA may be a natural or synthetic (engineered) transfer RNA.

The transfer RNA encoded by the nucleic acid may have a “normal” anticodon arm, i.e. an anticodon arm including 10 nucleotides forming the anticodon stem and 7 nucleotides forming the anticodon loop containing a three-base anticodon base-pairing with a respective codon on an mRNA. The anticodon can, for example, be designed to base-pair to a premature stop codon on an mRNA, and function as a suppressor tRNA. Alternatively, the encoded transfer RNA can, however, also have an extended anticodon loop having a four-base, five-base or six-base anticodon (see WO 2019/175316 A1, WO 2020/208169 A1, the disclosures of which are incorporated by reference herein in their entirety).

In an embodiment, the nucleic acid encoding the transfer RNA may also contain one or more introns. These intron(s) may be natural occurring introns. In preferred embodiments the intron(s) is (are) inserted into a nucleic acid encoding a tRNA that does not have an intron naturally. However, the tRNA, e.g., suppressor tRNA, encoded by the nucleic acid can also be a tRNA comprising a tRNA body of a tRNA the gene or pre-tRNA of which naturally contains one or more introns, which are, however, preferably different from the introns inserted in the nucleic acid according to the invention. Inclusion of one or more introns allows, at least in some applications, for close to natural tRNA transcription with factors (enzymes) that co-transcriptionally modify tRNAs which in turn leads to more stable output as functional tRNA.

In preferred embodiments, the encoded tRNA may be structurally modified in that the tRNA has a structurally modified D arm, anticodon arm, variable loop and/or T arm. “Structurally modified” means that the mature tRNA contains an anticodon arm, and/or a variable loop and/or a T-arm and/or a D arm that has been modified, i.e. has a nucleotide sequence, for example of the stem portion or the loop portion, that differs from the D arm and/or the anticodon arm and/or the variable loop and/or the T arm of a comparable naturally occurring tRNA. “Comparable” tRNAs are tRNA being aminoacylated by the same aminoacyl-tRNA synthetases. Particular preferred, the encoded tRNA contains, in its mature form, a D arm, an anticodon arm and/or a variable loop and/or a T arm that does not occur in natural tRNAs. It is to be noted here that the invention also relates to the tRNAs, as described below.

The anticodon arm of the mature tRNA encoded by the nucleic acid may, for example, have one of the following general structures with a modified stem portion:

5′-GCAGG-AC-Loop-CCTGT-3′
5′-TTGGG-AC-Loop-CTCAA-3′
5′-TTGGA-AC-Loop-TTCAA-3′
5′-ATGGT-AC-Loop-ACCAT-3′
5′-GCGGA-AC-Loop-TCCGC-3′
5′-GCGGT-AC-Loop-ACCGC-3′
5′-GGCGG-AC-Loop-CCGCC-3′
5′-GGCGC-AC-Loop-GCGCC-3′
5′-TTGGG-AC-loop-CCCAA-3′
5′-CTGGA-AC-loop-TCCAG-3′
5′-CCGGA-AC-loop-TCCGG-3′
5′-GCTGC-AC-loop-GCAGT-3′

Preferred anticodon (AC) arms of the encoded tRNA may have the following general sequences:

(SEQ ID NO: 135)
GCAGGNNNNNNNCCTGT
(SEQ ID NO: 136)
GCAGGNNNNNNNNCCTGT
(SEQ ID NO: 137)
GCAGGNNNNNNNNNCCTGT
(SEQ ID NO: 138)
GCAGGNNNNNNNNNNCCTGT
(SEQ ID NO: 139)
TTGGGNNNNNNNCTCAA
(SEQ ID NO: 140)
TTGGGNNNNNNNNCTCAA
(SEQ ID NO: 141)
TTGGGNNNNNNNNNCTCAA
(SEQ ID NO: 142)
TTGGGNNNNNNNNNNCTCAA
(SEQ ID NO: 143)
TTGGANNNNNNNTTCAA
(SEQ ID NO: 144)
TTGGANNNNNNNNTTCAA
(SEQ ID NO: 145)
TTGGANNNNNNNNNTTCAA
(SEQ ID NO: 146)
TTGGANNNNNNNNNNTTCAA
(SEQ ID NO: 147)
ATGGTNNNNNNNACCAT
(SEQ ID NO: 148)
ATGGTNNNNNNNNACCAT
(SEQ ID NO: 149)
ATGGTNNNNNNNNNACCAT
(SEQ ID NO: 150)
ATGGTNNNNNNNNNNACCAT
(SEQ ID NO: 151)
GCGGANNNNNNNTCCGC
(SEQ ID NO: 152)
GCGGANNNNNNNNTCCGC
(SEQ ID NO: 153)
GCGGANNNNNNNNNTCCGC
(SEQ ID NO: 154)
GCGGANNNNNNNNNNTCCGC
(SEQ ID NO: 155)
GCGGTNNNNNNNACCGC
(SEQ ID NO: 156)
GCGGTNNNNNNNNACCGC
(SEQ ID NO: 157)
GCGGTNNNNNNNNNACCGC
(SEQ ID NO: 158)
GCGGTNNNNNNNNNNACCGC
(SEQ ID NO: 159)
GGCGGNNNNNNNCCGCC
(SEQ ID NO: 160)
GGCGGNNNNNNNNCCGCC
(SEQ ID NO: 161)
GGCGGNNNNNNNNNCCGCC
(SEQ ID NO: 162)
GGCGGNNNNNNNNNNCCGCC
(SEQ ID NO: 163)
GGCGCNNNNNNNGCGCC
(SEQ ID NO: 164)
GGCGCNNNNNNNNGCGCC
(SEQ ID NO: 165)
GGCGCNNNNNNNNNGCGCC
(SEQ ID NO: 166)
GGCGCNNNNNNNNNNGCGCC

Underlined N's represent a 3 nt, 4 nt, 5 nt or 6 nt anticodon. N=A, C, G or T, or any modified base (in the final tRNA).

In preferred embodiments, the encoded tRNA comprises, for example, an anticodon (AC) arm of one of the following sequences:

(SEQ ID NO: 167)
GCAGGCTNNNAACCTGT
(SEQ ID NO: 168)
GCAGGCTNNNNAACCTGT
(SEQ ID NO: 169)
GCAGGCTNNNNNAACCTGT
(SEQ ID NO: 170)
GCAGGCTNNNNNNAACCTGT
(SEQ ID NO: 171)
TTGGGCTNNNAACTCAA
(SEQ ID NO: 172)
TTGGGCTNNNNAACTCAA
(SEQ ID NO: 173)
TTGGGCTNNNNNAACTCAA
(SEQ ID NO: 174)
TTGGGCTNNNNNNAACTCAA
(SEQ ID NO: 175)
TTGGACTNNNAATTCAA
(SEQ ID NO: 176)
TTGGACTNNNNAATTCAA
(SEQ ID NO: 177)
TTGGACTNNNNNAATTCAA
(SEQ ID NO: 178)
TTGGACTNNNNNNAATTCAA
(SEQ ID NO: 179)
ATGGTCTNNNAAACCAT
(SEQ ID NO: 180)
ATGGTCTNNNNAAACCAT
(SEQ ID NO: 181)
ATGGTCTNNNNNAAACCAT
(SEQ ID NO: 182)
ATGGTCTNNNNNNAAACCAT
(SEQ ID NO: 183)
GCGGACTNNNAATCCGC
(SEQ ID NO: 184)
GCGGACTNNNNAATCCGC
(SEQ ID NO: 185)
GCGGACTNNNNNAATCCGC
(SEQ ID NO: 186)
GCGGACTNNNNNNAATCCGC
(SEQ ID NO: 187)
GCGGTCTNNNAAACCGC
(SEQ ID NO: 188)
GCGGTCTNNNNAAACCGC
(SEQ ID NO: 189)
GCGGTCTNNNNNAAACCGC
(SEQ ID NO: 190)
GCGGTCTNNNNNNAAACCGC
(SEQ ID NO: 191)
GGCGGCTNNNAACCGCC
(SEQ ID NO: 192)
GGCGGCTNNNNAACCGCC
(SEQ ID NO: 193)
GGCGGCTNNNNNAACCGCC
(SEQ ID NO: 194)
GGCGGCTNNNNNNAACCGCC
(SEQ ID NO: 195)
GGCGCCTNNNAAGCGCC
(SEQ ID NO: 196)
GGCGCCTNNNNAAGCGCC
(SEQ ID NO: 197)
GGCGCCTNNNNNAAGCGCC
(SEQ ID NO: 198)
GGCGCCTNNNNNNAAGCGCC

The underlined N's represent a 3 nt, 4 nt, 5 nt or 6 nt anticodon. N=A, C, G or T, or any modified base. A preferred 3 nt anticodon is, for example, TCA for a tRNA acting as a PTC suppressor tRNA.

In a further preferred embodiment of the invention, the encoded tRNA comprises, in combination with one of the AC arms mentioned above, or alone, i.e. not in combination with one of the AC arms mentioned above, a variable loop (V loop) having one of the following sequences:

(SEQ ID NO: 199)
TGGGGTTTCCCC
(SEQ ID NO: 200)
AGGGGAAACCCC

In a further preferred embodiment of the invention, the encoded tRNA of the invention comprises, in combination with one of the AC arms described above and/or in combination with one of the V loops described above, or alone, i.e. not in combination with one of the AC arms mentioned above and/or not in combination with one of the V loops mentioned above, a T arm having the following general sequence:

GCGGG-T-Loop-CCCGT
ACGGG-T-Loop-CCCGT
GTAGG-T-Loop-CCCAT
GTCGG-T-Loop-CCCGT
GGCGG-T-Loop-CCGGT
GCAGG-T-Loop-CCCGT
GCCGG-T-Loop-CCGGT

Preferably, the T-Loop (positions 54-60 according to tRNA numbering convention) has the sequence HTCGANT, H being A, C, or T, i.e., CTCGANT, ATCGANT or TTCGANT, further preferred TTCGANT, for example TTCGAAT or TTCGAGT, preferably TTCGAGT.

In preferred embodiments, the encoded tRNA of the invention comprises, for example, a T arm of one of the following sequences

(SEQ ID NO: 201)
GCGGGTTCGAATCCCGT
(SEQ ID NO: 202)
ACGGGTTCGAATCCCGT
(SEQ ID NO: 203)
GCGGGTTCGAGTCCCGT
(SEQ ID NO: 204)
ACGGGTTCGAGTCCCGT
(SEQ ID NO: 205)
GTAGGTTCGAGTCCCAT
(SEQ ID NO: 206)
GTCGGTTCGAGTCCGAT
(SEQ ID NO: 207)
GCCGGTTCGAGTCCGGT
(SEQ ID NO: 208)
GCAGGTTCGAGTCCCGT
(SEQ ID NO: 209)
GCCGGTTCGAGTCCGGT

The loop part is in italics. The T arm has a stem of five base pairs.

In a further preferred embodiment of the invention, the encoded tRNA of the invention comprises, in combination with one of the AC arms described above and/or in combination with one of the V loops described above and/or in combination with one of the T arms described above, or alone, i.e. not in combination with one of the AC arms mentioned above and/or not in combination with one of the V loops mentioned above and/or not in combination with one of the T arms described above, an acceptor stem having the following general sequence:

5′-GGCTCTG-rest tRNA-CAGAGTC-3′
5′-GGCGCTG-rest tRNA-CAGCGTC-3′
5′-GGCGCGG-rest tRNA-CGGCGTC-3′

The term “rest tRNA” refers to the part of the tRNA between the 5′ and 3′ portions of the acceptor stem, and includes the D arm, V loops and T arm.

As mentioned above, for a given tRNA encoded by the synthetic DNA construct of the invention, the anticodon arm or at least the anticodon stem, the V loop, the T arm, or at least the T stem, and the acceptor stem can be independently selected from the anticodon arms/stems, V loops, T arms/stems and acceptor stems mentioned above. The encoded tRNA may thus only include one of the anticodon arms/stems, V loops, T arms/stems or acceptor stems mentioned above, for example only an anticodon arm with the sequence of SEQ ID NO: 171, or any combination thereof, for example an anticodon arm with the sequence of SEQ ID NO: 195 and a T arm having the sequence of SEQ ID NO: 203, or an anticodon arm with the sequence of SEQ ID NO: 171 and an acceptor stem as described above.

The skilled person is aware of the fact that a tRNA is aminoacylated with a specific amino acid by a specific aminoacyl tRNA synthetase (aaRS), and that the aaRS is able to recognize its cognate tRNA through unique identity elements at the acceptor stem and/or anticodon loop of the tRNA. In order to include in the DNA construct of the invention a tRNA gene of a tRNA which is, in its mature form, loaded with its cognate amino acid in vivo, the skilled person will design the encoded tRNA with suitable unique identity elements.

In the transfer RNA encoded in the synthetic DNA construct of the invention the anticodon loop may be extended by a large enough number of nucleotides to accommodate a four-, five- or six-base anticodon. The anticodon loop of a mature transfer RNA encoded by the synthetic vector of the invention may, for example, consist of 7-12, preferably 7 to 10 or 8-10, further especially preferred 8 or 9 nucleotides.

The synthetic DNA construct can, for example, be a synthetic vector, and thus be, for example, any suitable biological nucleic acid delivery vehicle, in particular a delivery vehicle suitable for delivery of a nucleic acid into a living mammalian cell, e.g., a human cell. Suitable vehicles include, for example, viral vectors such as adeno-associated virus (AAV)-based viral vectors, encapsulation in or coupling to nanoparticles, e.g., lipid nanoparticles, and others. Preferably, the vector is a viral vector. Viral vectors include, for example, adeno-associated (AAV) viruses, adenoviruses (e.g., AAV3, AAV8, or AAV9), retroviruses (see, for example, Bulcha, J. T., Wang, Y., Ma, H. et al. Viral vector platforms within the gene therapy landscape. Sig Transduct Target Ther 6, 53, 2021, doi: 10.1038/s41392-021-00487-6; Kotterman, M. A., Chalberg, T. W., Schaffer, D. V., 2015, Viral Vectors for Gene Therapy: Translational and Clinical Outlook, Annu. Rev. Biomed. Eng. 2015. 17:63-89, 10.1146/annurev-bioeng-071813-104938).

In a second aspect the invention relates to a pharmaceutical composition comprising the synthetic DNA construct according to the invention, and a pharmaceutically acceptable carrier. An example of a simple carrier is a buffer solution or a physiologic salt solution.

In a further aspect the invention relates to the synthetic DNA construct according to the first aspect of the invention, or the pharmaceutical composition according to the second aspect of the invention for use as a medicament. The synthetic DNA construct or pharmaceutical composition of the invention is especially useful for treating patients with a disease associated with a nonsense mutation, i.e. a premature termination codon (PTC), or a frameshifting causing the absence of a functional protein or the dysfunction of a protein. Examples for diseases, in which the tRNA of the invention may advantageously be employed are Hurler syndrome (MPS I, ICD code E76.0, beta-thalassemia (ICD-10 code D56.1), neurofibromatosis type 1 (NF1, ICD-10 code Q85.0), Duchenne muscular dystrophy (DMD, ICD-10 code G71.0, Crohn's disease (CD, ICD-10 code K50), cystic fibrosis (CF, ICD-10 code E84), neuronal ceroid lipofuscinosis (NCL, ICD-10 code E75.4) and Tay-Sachs disease (TSD, ICD-10 code E75.0).

The DNA construct, e.g., vector, of the invention is also useful for treating patients with a disease which is at least partly caused by the sequestration of a tRNA leading to depletion of the cellular pool of the tRNA, e.g. Charcot-Marie-Tooth disease (CMT disease, ICD-10 code DG600).

The DNA construct or pharmaceutical composition of the invention may, for example, also advantageously be used for treating a disease, e.g. hereditary motor and sensory neuropathy (Charcot-Marie-Tooth (CMT) disease), in which the natural tRNA is sequestered due to, for example, mutations in aminoacyl-tRNA-synthetase, and thus not or not sufficiently available for the translation machinery of the cell.

In a further aspect, the invention relates to a method of treating a person having a disease associated with a nonsense (PTC) or frameshift mutation, comprising administering an effective amount of the synthetic DNA construct of the invention or of a pharmaceutical composition comprising the synthetic DNA construct of the invention to the person. In a preferred embodiment the method is for treating Hurler syndrome (MPS I, ICD code E76.0), beta-thalassemia (ICD-10 code D56.1), neurofibromatosis type 1 (NF1, ICD-10 code Q85.0), Duchenne muscular dystrophy (DMD, ICD-10 code G71.0), Crohn's disease (CD, ICD-10 code K50), cystic fibrosis (CF, ICD-10 code E84), neuronal ceroid lipofuscinosis (NCL, ICD-10 code E75.4) or Tay-Sachs disease (TSD, ICD-10 code E75.0).

In a further aspect, the invention also relates to any of the structurally modified tRNAs, as described above, in the form of mature tRNA. In the tRNAs, the nucleotide thymine has to be replaced with the nucleotide Uracil.

In a still further aspect, the invention also relates to a synthetic DNA construct, consisting of or comprising one of the encoded modified tRNAs described above. Since the tRNAs are encoded in a DNA, the encoded tRNA may also be referred to as tDNA. Any T nucleotide of the tRNA in encoded form in the synthetic DNA construct will be replaced with a U nucleotide when the encoded tRNA is transcribed. The synthetic DNA construct comprises or consists of a DNA encoding a tRNA structurally modified in that the tRNA, i.e., the tRNA resulting from transcription of the synthetic construct, has a structurally modified D arm, anticodon arm, variable loop and/or T arm. The synthetic DNA construct may include regulatory elements, e.g., 5′ leader sequences comprising promoters and the like, other than those described above for the synthetic DNA construct according to the first aspect of the invention, affecting, for example, transcription. The DNA construct may be a synthetic vector, e.g., expression vector for the delivery and expression of the tRNA in a cell, e.g., human cell.

The anticodon arm of the tRNA encoded by the construct, for example may have one of the following general structures with a modified stem portion (see above):

5′-GCAGG-AC-Loop-CCTGT-3′
5′-TTGGG-AC-Loop-CTCAA-3′
5′-TTGGA-AC-Loop-TTCAA-3′
5′-ATGGT-AC-Loop-ACCAT-3′
5′-GCGGA-AC-Loop-TCCGC-3′
5′-GCGGT-AC-Loop-ACCGC-3′
5′-GGCGG-AC-Loop-CCGCC-3′
5′-GGCGC-AC-Loop-GCGCC-3′
5′-TTGGG-AC-loop-CCCAA-3′
5′-CTGGA-AC-loop-TCCAG-3′
5′-CCGGA-AC-loop-TCCGG-3′
5′-GCTGC-AC-loop-GCAGT-3′

Preferred anticodon arms of the encoded tRNA may have one of the sequences of SEQ ID NO: 135-198 (see above):

In further preferred embodiments of the invention, the encoded tRNA comprises, in combination with one of the AC arms mentioned above, or alone, i.e. not in combination with one of the AC arms mentioned above, a variable loop (V loop) having one of the following sequences of SEQ ID NO: 199-200 (see above).

In a further preferred embodiment of the invention, the encoded tRNA of the invention comprises, in combination with one of the AC arms described above and/or in combination with one of the V loops described above, or alone, i.e. not in combination with one of the AC arms mentioned above and/or not in combination with one of the V loops mentioned above, a T arm having the following general sequence (see above):

GCGGG-T-Loop-CCCGT
ACGGG-T-Loop-CCCGT
GTAGG-T-Loop-CCCAT
GTCGG-T-Loop-CCCGT
GGCGG-T-Loop-CCGGT
GCAGG-T-Loop-CCCGT
GCCGG-T-Loop-CCGGT

Preferably, the T-Loop (positions 54-60 according to tRNA numbering convention) has the sequence HTCGANT, H being A, C, or T, i.e., CTCGANT, ATCGANT or TTCGANT, further preferred TTCGANT, for example TTCGAAT or TTCGAGT, preferably TTCGAGT.

In preferred embodiments, the encoded tRNA of the invention comprises, for example, a T arm of one of the sequences of SEQ ID NO: 201-209.

In a further preferred embodiment of the invention, the tRNA encoded in the synthetic DNA construct according to this aspect of the invention comprises, in combination with one of the AC arms described above and/or in combination with one of the V loops described above and/or in combination with one of the T arms described above, or alone, i.e. not in combination with one of the AC arms mentioned above and/or not in combination with one of the V loops mentioned above and/or not in combination with one of the T arms described above, an acceptor stem having the following general sequence:

5′-GGCTCTG-rest tRNA-CAGAGTC-3′
5′-GGCGCTG-rest tRNA-CAGCGTC-3′
5′-GGCGCGG-rest tRNA-CGGCGTC-3′

The term “rest tRNA” refers to the part of the tRNA between the 5′ and 3′ portions of the acceptor stem, and includes the D arm, V loops and T arm. Of course, the “rest tRNA” is not to be considered a part of the acceptor stem.

In a preferred embodiment, the synthetic DNA construct of the invention consists of or comprises a nucleic acid encoding a transfer RNA, the encoded transfer RNA comprising
a) an anticodon arm having or comprising one of the sequences of SEQ ID NO: 135-198, and/or
b) a variable loop having or comprising one of the sequences of SEQ ID NO: 199-200, and/or
c) a T arm having or comprising one of the sequences according to SEQ ID NO: 201-209, and/or
d) an acceptor stem having the structure 5′-GGCTCTG-rest tRNA-CAGAGTC-3′ or 5′-GGCGCTG-rest tRNA-CAGCGTC-3′.

The “rest tRNA” is not to be considered a part of the acceptor stem.

It should be noted that a reference to a tRNA structure (e.g., T arm, AC arm etc.) within the DNA construct is to be understood as a reference to the DNA sequence(s) encoding the structure in the DNA construct. When transcribed into a mature tRNA, the sequences will be transcribed into RNA sequences forming the structure in the mature tRNA.

In a further aspect the invention relates to a pharmaceutical composition comprising the synthetic DNA construct according to the invention, as described immediately above this paragraph and a pharmaceutically acceptable carrier. An example of a simple carrier is a buffer solution or a physiologic salt solution.

The invention will be described in the following by way of examples and the appended figures for illustrative purposes only.

FIG. 1. Schematic drawing of a generalized “consensus” tRNA structure and its numbering according to tRNA numbering convention.

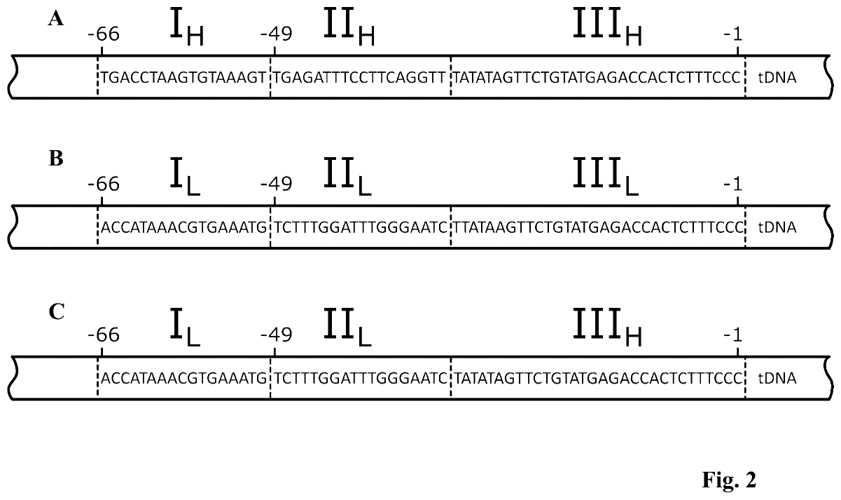

FIG. 2. Schematic drawing of part of three example embodiments of the first embodiment of a synthetic DNA construct of the invention.

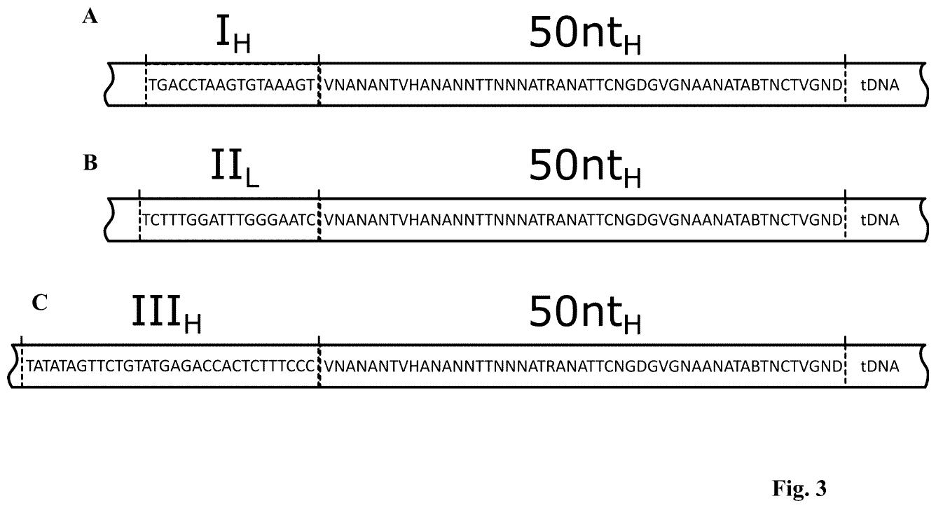

FIG. 3. Schematic drawing of part of three example embodiments of the second embodiment of a synthetic DNA construct of the invention.

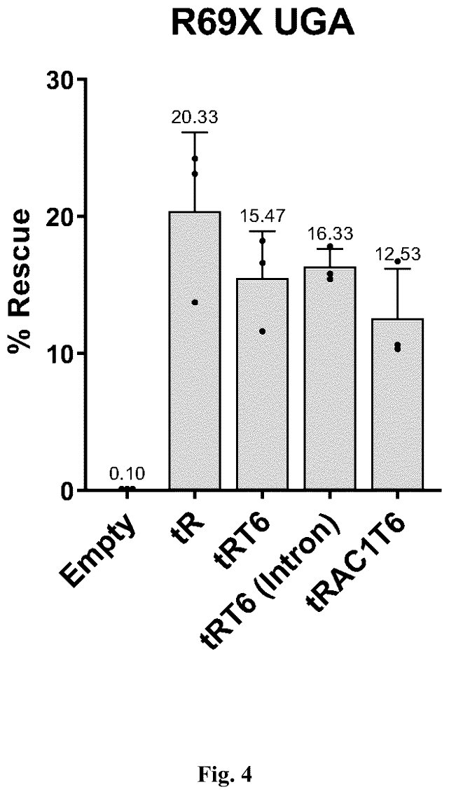

FIG. 4. Rescue activity of various suppressor tRNA variants.

FIG. 1 depicts an example of a tRNA numbered according to the conventional numbering applied to a generalized “consensus” tRNA, beginning with 1 at the 5′ end and ending with 76 at the 3′ end. The tRNA is composed of tRNA nucleotides 11 and has the common cloverleaf structure of tRNA comprising an acceptor stem 2 with the CCA tail 10, a T arm 3 with the TψC loop 6, a D arm 4 with the D loop 7 and an anticodon arm 5 with a five-nucleotide stem portion 8 and the anticodon loop 9. In such a “consensus” tRNA the nucleotides of the natural anticodon triplet 25 is always at positions 34, 35 and 36, regardless of the actual number of previous nucleotides. A tRNA may, for example, contain additional nucleotides between positions 1 and 34, e.g. in the D loop 7, and in the variable loop 24 between positions 45 and 46. Additional nucleotides may be numbered with added alphabetic characters, e.g. 20a, 20b etc. In the variable loop 24 the additional nucleotides are numbered with a preceding “e” and a following numeral depending on the position of the nucleotide in the loop. Positions of modified nucleotides within a tRNA of the invention are marked as black filled circles.

FIG. 2 schematically shows part of three example embodiments of a first embodiment of a synthetic DNA construct of the invention including the sequence motifs I_{H}, I_{L}, II_{H}, II_{L}, III_{H} and III_{L} in three different combinations. FIG. 2A shows an embodiment of a DNA construct including the sequence motifs I_{H}, II_{H} and III_{H} (P1, SEQ ID NO: 12), FIG. 2B shows an embodiment of a vector including the sequence motifs I_{L}, II_{L} and III_{L} (P2, SEQ ID NO: 13), and FIG. 2C shows an embodiment of a vector including the sequence motifs I_{L}, II_{L} and III_{H} (P23, SEQ ID NO: 34).

FIG. 3 schematically shows part of three example embodiments of the second embodiment of a synthetic DNA construct of the invention. In these embodiments, the sequence motifs I_{H}, II_{L} and III_{H}, are each combined with the sequence motifs 50 nt_{H}. In FIG. 3A, the sequence motif I_{H} is combined with the sequence motif 50 nt_{H} (SEQ ID NO: 51), in FIG. 3B the sequence motif II_{L} is combined with the sequence motif 50 nt_{H} (SEQ ID NO: 59), and in FIG. 3C the sequence motif III_{H} is combined with the sequence motif 50 nt_{H} (SEQ ID NO: 55).

FIG. 4 shows the rescue activity of various suppressor tRNA variants. tRNA variants embedded in a separate vector were co-transfected with a vector encoding firefly luciferase with a PTC (TGA) in place of an arginine codon (CGA, “R69X UGA”) in HEK293 cells. The luciferase expression was measured 24 h post-transfection and the tRNA suppression activity (% rescue) is presented as percentage of the wild-type luciferase. The following suppressor tRNAs were considered: (i) tR, (ii) tRT6, (iii) tRT6 retaining an intron sequence and (iv) tRACIT6. Mock transfected cells (empty) served as negative control. For this, cells were transfected with an empty vector without luciferase, but subjected to the same transfection procedure, i.e. treated with same amounts of lipofectamine which alters slightly cell growth.

To test readthrough efficiency of suppressor tRNA a reporter plasmid was used with Firefly luciferase (FLuc) driven by EF1a, to achieve the desired arginine PTC, the 69th codon of FLuc in the reporter which is arginine was mutated to a stop codon UGA, embedded in pTwist EF1 Alpha Puro plasmid backbone. The tRNA was also encoded using a plasmid system in which the desired suppressor tRNA as its DNA form, some of them retaining the intron, driven by U6 promoter embedded in pcDNA3.1 plasmid backbone.

HEK293 cells were seeded in 96-well cell culture plates at 1×10⁴ cells/well and grown in Dulbecco's Modified Essential Medium (DMEM, Pan Biotech) supplemented with 10% fetal bovine serum (FBS, Pan Biotech) and 2 mM L-glutamine (Thermo Fisher Scientific). 16 to 24 hours later, cells were co-transfected in triplicate with 25 ng R69X PTC-FLuc or WT Fluc plasmids and 100 ng of each suppressor tRNA variant or empty control plasmids using lipofectamine 3000 (Thermo Fisher Scientific). After four to six hours, medium was replaced and 24 hours post-transfection cells were lysed with 1× passive lysis buffer (Promega) and luciferase activity measured with luciferase assay system (Promega) and Spark microplate reader (Tecan). Readthrough activity was presented as percentage of the activity of WT luciferase that was expressed on a separate expression vector.

First, the anticodon of tRNA^{Arg} (TCT 3-1, source tRNA data base:
http://gtrnadb.ucsc.edu/genomes/eukaryota/Hsapi38/) was exchanged to pair to UGA PTC, thus creating tR (FIG. 3). We next changed the TψC stem (creating tRT5 variant) and simultaneously TψC stem and acceptor stem (tRAC1T6 variant) in tR. In addition, the natural intron of tRNA^{Arg} was kept (variant tRT6 (intron)), as introns usually provide an expression benefit to tRNAs. For the activity screen, all tRNA variants were cloned into the plasmid (pTwist EF1 Alpha Puro) and co-transfected in HEK293 cells together with a plasmid bearing the firefly luciferase (FLuc) reporter with arginine PTC (R69X). After 24 h, the luciferase activity was measured and normalized to that of the wildtype luciferase (% rescue, FIG. 3). All tRNA variants efficiently restored the expression of R69X luc in a range of 12-20% (of the wildtype luciferase; numbers displayed in FIG. 3 (n=3 independent biological replicates) are mean values of restored luciferase activity).

The sequences of the tRNA mentioned above are as follows (anticodon underlined; T stem, AC stem, T stem and acceptor stem double underlined; intron in lower case letters):

tR (based on native tRNA-Arg-TCT-3-1 (Arg-chr9.tRNA5),
replaced UCA anticodon, 5′-3′;
SEQ ID NO: 210):
GGCTCTGTGGCGCAATGGATAGCGCATTGGACTTCAAATTCAAAGGTTGTGGGTTC
GAGTCCCACCAGAGTCG
RT6 (Arg-chr9.tRNA5 with modified T-stem, 5′-3′; SEQ ID NO: 211):
GGCTCTGTGGCGCAATGGATAGCGCATTGGACTTCAAATTCAAAGGTTGCGGGTTC
GAGTCCCGTCAGAGTCG
RT6 (Intron) (Arg-chr9.tRNA5 maintaining the intron
and with modified T-stem, 5′-3′;
SEQ ID NO: 212):
GGCTCTGTGGCGCAATGGATAGCGCATTGGACTTCAAgctgagcctagtgtggtcATTCAAAG
GTTGCGGGTTCGAGTCCCGTCAGAGTCG
tRAc1T6 (tRT6 with changes in the acceptor stem; SEQ ID NO: 213):
GGCGCTGTGGCGCAATGGATAGCGCATTGGACUTCAAATTCAAAGGTTGCGGGTTC
GAGTCCCGTCAGCGTCG

The positions of the D stem, anticodon stem, T stem and acceptor stem in the above tRNA are as follows (anticodon 34 . . . 36):

Sequences without intron (SEQ ID NO: 127, 128, 130)

| | 5′ part | 3′ part |
|---|---|---|
| Acceptor stem: | 1 . . . 7; | 66 . . . 72 |
| D stem: | 10 . . . 13; | 22 . . . 25 |
| Anticodon stem: | 27 . . . 31; | 39 . . . 43 |
| T stem: | 49 . . . 53 | 61 . . . 65 |

Sequence with intron (SEQ ID NO: 129; intron: 38 . . . 55)

| | 5′ part | 3′ part |
|---|---|---|
| Acceptor stem: | 1 . . . 7; | 84 . . . 90 |
| D stem: | 10 . . . 13; | 22 . . . 25 |
| Anticodon stem: | 27 . . . 31; | 57 . . . 61 |
| T stem: | 67 . . . 71 | 79 . . . 83 |

1. A synthetic DNA construct comprising (A) a nucleic acid encoding a transfer RNA and (B) a 5′ leader sequence, the 5′ leader sequence comprising at least one of:
a) a sequence motif selected from the group
consisting of the sequence motifs
(SEQ ID NO: 1)
TGACCTAAGTGTAAAGT,
I_{H},
(SEQ ID NO: 2)
TGAGATTTCCTTCAGGTT, II_{H},
(SEQ ID NO: 3)
TATATAGTTCTGTATGAGACCACTCTTTCCC,
III_{H},
(SEQ ID NO: 4)
ACCATAAACGTGAAATG, I_{L},
(SEQ ID NO: 5
TCTTTGGATTTGGGAATC, II_{L},
and
(SEQ ID NO: 6)
TTATAAGTTCTGTATGAGACCACTCTTTCCC;
III_{L},
[[and/or]]
b) a sequence motif selected from the group
consisting of the sequence motifs
(SEQ ID NO: 7)
VNANANTVHANANNTTNNNATRANATTCNGDGV
GNAANATABTNCTVGND,
50nt_{H},
and
(SEQ ID NO: 8)
GCNGGDGGCGNGTTCBBCTGTNVNAGCNTNCG
DNGNCGTTTNNCNTCGGT;
50nt_{L},
and
c) a sequence motif selected from the group
consisting of the sequence motifs
(SEQ ID NO: 9)
GAAATGCCTT,
10nt_{H1},
(SEQ ID NO: 10)
GTGGGAACTA
10nt_{H2},
and [[/or]]
(SEQ ID NO: 11)
GTGTTGCTTG.
10nt_{H3},

2. The synthetic DNA construct according to claim 1, the 5′ leader sequence comprising one of the sequence motifs of feature b) above, with the proviso that the 5′ leader sequence does not comprise one of the sequences of SEQ ID NO: 39, 42 or 46; or, if the 5′ leader sequence comprises one of the sequences of SEQ ID NO: 39 or 46, the encoded transfer RNA is not a tRNA^{Gly}, and, if the 5′ leader sequence comprises the sequence of SEQ ID NO: 42, the encoded transfer RNA is not a tRNA^{Leu}.

3. The synthetic DNA construct according to claim 1, wherein,
aa) in relation to a) above,
i. the 5′ leader sequence does not comprise the sequence motif I_{H} if it comprises the sequence motif I_{L}, and vice versa,
ii. the 5′ leader sequence does not comprise the sequence motif II_{H} if it comprises the sequence motif II_{L}, and vice versa, and
iii, the 5′ leader sequence does not comprise the sequence motif III_{H} if it comprises the sequence motif III_{L}, and vice versa; or
bb) in relation to b) above, the 5′ leader sequence does not comprise the sequence motif 50 nt_{H} if it comprises the sequence motif 50 nt_{L}, and vice versa.

4. The synthetic DNA construct according to claim 1, wherein,
aa) in relation to a) above,
i. if the 5′ leader sequence comprises the sequence motif I_{H} or I_{L}, the sequence motif I_{H} or I_{L} has, in 5′-3′ direction, the positions-66 to −50,
ii. if the 5′ leader sequence comprises the sequence motif II_{H} or II_{L}, the sequence motif II_{H} or II_{L} has, in 5′-3′ direction, the positions-49 to −32, and
iii. if the 5′ leader sequence comprises the sequence motif III_{H} or III_{L}, the sequence motif III_{H} or III_{L} has, in 5′-3′ direction, the positions-31 to −1; or
bb) in relation to b) above, the sequence motif 50 nt_{H}, or 50 nt_{L} has, in 5′-3′ direction, the positions-50 to −1.

5. The synthetic DNA construct according to claim 1, wherein the 5′ leader sequence comprises,
aa) in relation to a) above, at least two sequence motifs selected from the sequence motifs I_{L}, II_{L}, III_{L}, I_{H}, II_{H} or III_{H}, and wherein the at least two sequence motifs are arranged, in 5′-3′ direction, in the order I_{H}-II_{H}, II_{H}-III_{H}, I_{H}-III_{H}, I_{L}-II_{L}, II_{L}-III_{L}, I_{L}-III_{L}, I_{H}-II_{L}, I_{H}-III_{L}, II_{H}-III_{L}, I_{L}-II_{H}, II_{L}-III_{H}, or I_{L}-III_{H}; or
cc) in relation to c) above, at least two sequence motifs of the sequence motifs 10 nt_{H1}, 10 nt_{H2}, and 10 nt_{H3}, the at least two sequence motifs being:
i. GAAATGCCTT, 10 nt_{H1}, (SEQ ID NO: 9) and GTGTTGCTTG, 10 nt_{H3}, (SEQ ID NO:11), wherein the sequence motifs are arranged, in 5′-3′ direction, in the following order:
10 nt_{H3}-10 nt_{H1}, or
ii. GAAATGCCTT, 10 nt_{H1}, (SEQ ID NO: 9) and GTGGGAACTA, 10 nt_{H2}, (SEQ ID NO:10), wherein the sequence motifs are arranged, in 5′-3′ direction, in the following order:
10 nt_{H1}-10 nt_{H2}, or
iii. GTGGGAACTA, 10 nt_{H2}, (SEQ ID NO:10), and GTGTTGCTTG, 10 nt_{H3}, (SEQ ID NO:11), wherein the sequence motifs are arranged, in 5′-3′ direction, in the following order:
10 nt_{H3}-10 nt_{H2}.

6. The synthetic DNA construct according to claim 1, wherein the 5′ leader sequence comprises
aa) in relation to a) above, three sequence motifs selected from the sequence motifs I_{L}, II_{L}, III_{L}, I_{H}, II_{H} or III_{H}, preferably in the following order, in 5′-3′ direction: I_{L}-II_{L}-III_{L}, I_{H}-II_{H}-III_{H}, I_{H}-II_{L}-III_{L}, I_{L}-II_{H}-III_{L}, I_{L}-II_{L}-III_{H}, I_{H}-II_{H}-III_{L}, I_{H}-II_{L}-III_{H}, or I_{L}-II_{H}-II_{H}; or
cc) in relation to c) above all three sequence motifs 10 nt_{H1}, 10 nt_{H2}, and 10 nt_{H3}.

7. The synthetic DNA construct according to claim 1, wherein the 5′ leader sequence comprises one of the sequence motifs selected from the sequence motifs I_{H}, II_{H} III_{H}, I_{L}, II_{L}, and III_{L} of feature a) above, followed immediately, in 5′-3′ direction, by one of the sequence motifs selected from the sequence motifs 50 nt_{L} and 50 nt_{H} of feature b) above.

8. The synthetic DNA construct according to claim 7, wherein the 5′ leader sequence comprises or has one of the sequences of SEQ ID NO: 51-62.

9. The synthetic DNA construct according to claim 7, wherein the 5′ leader sequence comprises one of the sequence motifs I_{H}, II_{H}, III_{H}, followed immediately, in 5′-3′ direction, by the sequence motif 50 nt_{H}; or comprises one of the sequence motifs I_{L}, II_{L}, III_{L}, followed immediately, in 5′-3′ direction, by the sequence motif 50 nt_{L}.

10. The synthetic DNA construct according to claim 1, wherein the 5′ leader sequence comprises i) one of the sequence motifs selected from the sequence motifs I_{H}, II_{H} III_{H}, I_{L}, II_{L}, and III_{L} of feature a) above, followed immediately, in 5′-3′ direction, by a spacer region comprising or composed of 10-30 repeats of a spacer sequence of 8-12, preferably 9-11, more preferably 10 nucleotides, followed immediately, in 5′-3′ direction, by one of the sequence motifs selected from the sequence motifs 50 nt_{L} and 50 nt_{H} of feature b) above, or ii) one of the sequence motifs selected from the sequence motifs I_{H}, II_{H} III_{H}, I_{L}, II_{L}, and III_{L} of feature a) above, flanked, at its 5′ end, by a spacer region comprising or composed of 10-30 repeats of a spacer sequence of 8-12, and at its 3′ end, by one of the sequence motifs selected from the sequence motifs 50 nt_{L} and 50 nt_{H} of feature b) above.

11. The synthetic DNA construct according to claim 1, wherein, if the nucleic acid encodes a naturally occurring transfer RNA, the 5′ leader sequence is not from said naturally occurring transfer RNA.

12. The synthetic DNA construct according to claim 1, wherein the 5′ leader sequence has or comprises aa) in relation to a) above, a sequence according to one of SEQ ID NO: 12-37 bb) in relation to b) above, a sequence according to one of SEQ ID NO: 39-50.

13. The synthetic DNA construct according to claim 1, further comprising the coding sequence for a natural or synthetic transfer RNA comprising a B box comprising the sequence H₅₄T₅₅C₅₆G₅₇A₅₈N₅₉T₆₀, the index representing the positions of the nucleotides in the transfer RNA, the position numbering following transfer RNA numbering convention.

14. The synthetic DNA construct according to claim 13, wherein the 5′ leader sequence comprises one of the sequence motifs 50 nt_{H} or 50 nt_{L} of feature b) above, wherein, if the 5′ leader sequence comprises the sequence motif 50 nt_{L}, it immediately follows a sequence motif selected from the sequence motifs I_{L}, II_{L}, and III_{L}, and wherein, if the 5′ leader sequence comprises the sequence motif 50 nt_{H}, it immediately follows a sequence motif selected from the sequence motifs I_{H}, II_{H}, and III_{H}.

15. The synthetic DNA construct according to claim 14, wherein the 5′ leader sequence does not comprise one of the sequences of SEQ ID NO: 39, 42 or 46; or, if the 5′ leader sequence comprises one of the sequences of SEQ ID NO: 39 or 46, the encoded transfer RNA is not a tRNA^{Gly}, and, if the 5′ leader sequence comprises the sequence of SEQ ID NO: 42, the encoded transfer RNA is not a tRNA^{Leu}.

16. The synthetic DNA construct according to claim 1, the tRNA encoded by the nucleic acid comprising:
a) an anticodon arm having or comprising one of the sequences of SEQ ID NO: 135-198, and/or
b) a variable loop having or comprising one of the sequences of SEQ ID NO: 199-200, and/or
c) a T arm having or comprising one of the sequences according to SEQ ID NO: 201-209, and/or
d) an acceptor stem having the structure 5′-GGCUCUG-rest tRNA-CAGAGUC-3′ or 5′-GGCGCUG-rest tRNA-CAGCGUC-3′.

17. The synthetic DNA construct according to claim 1, wherein the synthetic DNA construct is a synthetic vector.

18. A pharmaceutical composition comprising the synthetic DNA construct according to claim 1 and a pharmaceutically acceptable carrier.

19. (canceled)

20. A medicament comprising the synthetic DNA construct according to claim 1 for treatment of a disease which is at least partly caused by a nonsense or frameshift mutation leading to the production of a protein being dysfunctional or non-functional compared to the wild-type protein, or at least partly caused by the intracellular sequestration of a tRNA leading to depletion of the cellular pool of the tRNA.

21. The medicament according to claim 20 for use as a medicament for treating Hurler syndrome, beta-thalassemia, Crohn's disease, Tay-Sachs disease, Duchenne muscular dystrophy, cystic fibrosis, neuronal ceroid lipofuscinosis, neurofibromatosis type 1, or Charcot-Marie-Tooth disease.

22. A synthetic DNA construct consisting of a nucleic acid encoding a transfer RNA, the encoded transfer RNA comprising at least one of
a) an anticodon arm having or comprising one of the sequences of SEQ ID NO: 135-198,
b) a variable loop having or comprising one of the sequences of SEQ ID NO: 199-200,
c) a T arm having or comprising one of the sequences according to SEQ ID NO: 201-209, and
d) an acceptor stem having the structure 5′-GGCTCTG-rest tRNA-CAGAGTC-3′ or 5′-GGCGCTG-rest tRNA-CAGCGTC-3′.
